(19) United States
(12) Patent Application Publication
(10) Pub. No.: US 2023/0298717 A1
(43) Pub. Date: Sep. 21, 2023

(54) SYSTEMS AND METHODS FOR REMOTE MANAGEMENT OF MONITORED EXERCISE AND REHABILITATION WITH EMERGENCY RESPONSES

(71) Applicant: Kivo Health, Inc., Oakland, CA (US)

(72) Inventors: Victor Jonas Sadauskas, Oakland, CA (US); Vaughn Michael Koch, Oakland, CA (US)

(21) Appl. No.: 18/165,919

(22) Filed: Feb. 7, 2023

Related U.S. Application Data
(60) Provisional application No. 63/418,329, filed on Oct. 21, 2022.
(60) Provisional application No. 63/308,887, filed on Feb. 10, 2022.

(51) Int. Cl. G16H 20/00; A61B 5/00; A61B 5/11; A61B 5/024; G16H 10/60

(52) U.S. Cl. CPC G16H 20/00; A61B 5/02416; A61B 5/1112; A61B 5/7203; A61B 5/747; G16H 10/60

(57) ABSTRACT

Systems and methods for the remote management of monitored exercise and rehabilitation in the setting of respiratory conditions are provided. Particularly, therapy guidance to an agent is recited. Sensory data and photoplethysmography (PPG) data are collected from a patient. The sensory data is discarded when the PPG data differs from a prototypical waveform pattern. The remaining sensory data is analyzed, in light of historical data, to generate recommendations for the patient. Additionally, systems and methods for providing an emergency services response during the therapy session is provided. The location of the patient is assessed. The system then converts the location information into longitude/latitude coordinates or into jurisdictional named entities. The translated location data for the patient may then be cross referenced with a real-time updated database of emergency medical services (EMS) contact information. In the event of an emergency situation, this data can then be provided to the healthcare worker.

### CROSS REFERENCE TO RELATED APPLICATIONS

This non-provisional application claims the benefit and priority of U.S. Provisional Application No. 63/308,887 (Attorney Docket BWV-2201-P), filed on Feb. 10, 2022, and U.S. Provisional Application No. 63/418,329 (Attorney Docket BWV-2202-P), filed on Oct. 21, 2022, both pending and of the same title, which applications are incorporated herein in their entirety by this reference.

### BACKGROUND

The present invention relates in general to the field of management emergency responses in the context of respiratory condition therapies (including but not limited to chronic obstructive pulmonary disease (COPD), asthma, interstitial lung disease (ILD), cystic fibrosis (CF), long COVID, etc.) and more specifically to remote handling of respiratory rehabilitation and exercise regimens and the generation of emergency reactions during such sessions. Such systems and methods are useful for improving patient outcomes in the event of an emergency, or suspected emergency, caused by, exacerbated by, or simply during respiratory therapy.

Respiratory conditions are a significant health issue. They refer to a group of diseases that cause airflow blockage, inflammation, increased secretions, breathing-related problems, fibrosis, and poor oxygen and carbon dioxide exchange at the alveolar level, including emphysema, chronic bronchitis, asthma, interstitial lung disease (ILD), cystic fibrosis (CF), long COVID and other respiratory conditions. Looking at only a subset of respiratory conditions, COPD, at least 16 million Americans have been diagnosed with COPD, and millions more people suffer from COPD, but have not been diagnosed and are not being treated. Although there is no cure for COPD, it can be managed.

One of the most effective treatments for respiratory conditions is a consistent and highly regimented set of exercise therapies. These therapies are generally done in the context of a structured environment, monitored by a specialist. This is because too much strain on the respiratory conditions patient can be detrimental, and yet too little activity can lose its therapeutic value. Secondly, by having a monitored therapy session, there is an increased degree of compliance on behalf of the patient. Consistency and frequency of these therapies are instrumental in providing lasting and impactful improvements in patients with respiratory conditions.

However, there are hurdles to in-person, frequent respiratory condition exercise therapies. Firstly, they can be expensive, as the agent/therapist has to travel to the patient’s home, or to a therapy center, and must focus their attention on a relatively few patients with respiratory conditions at a time (e.g., 6-8 patients per session). Secondly, for instances where the therapies are held outside of the patient’s home, there are issues of transportation and other logistical issues for the patient to travel to the therapy center, which may be very difficult for many patients, or many patients simply do not want to travel for therapy. Ultimately, these various hurdles reduce the frequency and/or compliance with the respiratory condition exercise regimen. This can drastically reduce the efficacy of the therapy.

One attempt to rectify these issues is to have the patient perform at-home, non-supervised exercises. While these exercise regiments help marginally in managing respiratory condition symptoms, they generally have significantly lower efficacy than a monitored therapy session, as they are not adjusted on-the-fly based upon the needs of the patient. Unsupervised exercise regimens also raise safety concerns, as some people with respiratory conditions desaturate rapidly upon engaging in physical activity. Persistent exercise while the blood oxygen content is below an acceptable threshold may place strain on the cardiovascular system and actually result in worsening overall health.

Additionally, patients receiving remote therapy are generally at an increased risk of suffering complications during periods of increased physical strain. When the therapies are in person with the respiratory therapist (or other healthcare worker), the healthcare worker is readily available to assist the patient, and when needed, provide emergency care needs (contacting emergency medical services and the like). In the case of remote treatments, these interventions are not as trivial, and as such, integrated means for the management of emergency responses into such remote systems is a significant need.

As such, it is therefore apparent that an urgent need exists for systems and methods for enabling remote and efficient respiratory conditions managed therapies. Such systems and methods are designed to provide the patient with more efficacious respiratory condition symptom management. Further, systems and methods are designed to provide the patient with more efficacious respiratory condition symptom management while still maintaining a degree of safety that exists when engaging in in-person therapy sessions.

### SUMMARY

The present systems and methods relate to improving respiratory conditions (e.g., chronic obstructive pulmonary disease (COPD), asthma, interstitial lung disease (ILD), cystic fibrosis (CF), long COVID, etc.) therapies. In particular, the present systems and methods improve the rehabilitation and exercise regimens of respiratory condition patients that are supervised by a agent (virtual or otherwise). In some embodiments the agent may be a respiratory therapist, a physical therapist, a coach, a clinician, trainer, or similar individual. Such systems and methods enable improvements in the patient’s overall health, and management of their respiratory condition symptoms.

In some embodiments, systems and methods for providing therapy guidance to an agent are recited. These systems and methods include collecting sensory data and photoplethysmography (PPG) data from a patient. In some cases, the sensory data is heartrate and blood oxygen saturation. The sensory data may also include perfusion index (PI) percentage, blood pressure, electrocardiogram (ECG) signals, audio and video signals, body temperature, and carbon dioxide levels in the patient’s exhalation.

The sensory data may be discarded when the PPG data differs from a prototypical waveform pattern by a configured threshold. The prototypical waveform pattern is a saw-toothed shape, and the PPG data differing from the prototypical waveform pattern is calculated by a least means squared algorithm.

The collected sensory data is analyzed, in light of historical data, to generate recommendations for the patient. The historical data may include patient profile data as well as past sensory data. The recommendations may be generated by either rule based models or machine learning (ML) models. In some embodiments, the recommendations may include real-time recommendations which include altering exercise levels to make the measured physiological data reach a target range. The recommendation is supplied to the agent as a pop-up alert. In some particular situations the agent may be a virtual agent. In some embodiments, the recommendations may also be performed over time using historical data. These recommendations may be provided outside of sessions (or during, depending upon recommendation type). In some cases, recommendations may be formulated using not only the collected sensory data (real-time and historical), but also non-health data sources such as barometric pressure, air quality, ambient temperature, and the like.

Further present systems and methods relate to improving safety and emergency response management when patients are engaged in such remote respiratory therapies. In particular, the present systems and methods improve the ability to identify emergency situations (or situations where a lesser intervention is needed), and react appropriately and efficiently to such emergencies.

In some embodiments, systems and methods for providing an emergency services response during a remote therapy session includes the initiation of the remote therapy session between the healthcare worker and the patient. In some cases, the healthcare worker is a human therapist, and in other situations the healthcare worker may be a virtual therapist. It should be noted that while the term “therapist” is used extensively herein, any healthcare worker may be considered under this disclosure. For example, the patient could be working with a physical therapist, registered nurse, doctor, physician’s assistant, or other healthcare worker. The location of the patient is assessed. This can include the collection of the patient’s residence in advance, and then confirming their location in real time. It may also include using GPS information, cellular tower triangulation, and direct confirmation (verbal or otherwise) by the patient. The system then converts the location information into longitude/latitude coordinates or into jurisdictional named entities. The translated location data for the patient may then be cross referenced with a real-time updated database of emergency medical services (EMS) contact information. In the event of an emergency situation, this data can then be provided to the healthcare worker. The EMS includes an emergency dispatcher, local hospitals, ambulatory services, police and fire department.

In some cases, sensory data is collected from the patient. Sensory data can include any of pulse rate, blood oxygen saturation, photoplethysmography (PPG) data, a perfusion index (PI) percentage, blood pressure, electrocardiogram (ECG) signals, audio and video signals, and temperature. This sensory data may be monitored and used to identify when an emergency is happening. This may be by the healthcare worker initiating the emergency trigger, the patient initiating it verbally or by selecting a “panic” button on the virtual therapy system or may be generated by a machine learning algorithm that consumes the sensory data and patient history data. In some cases, a medical director may be brought into the remote therapy session and may triage the situation and trigger the emergency response.

Note that the various features of the present invention described above may be practiced alone or in combination. These and other features of the present invention will be described in more detail below in the detailed description of the invention and in conjunction with the following figures.

### BRIEF DESCRIPTION OF THE DRAWINGS

In order that the present invention may be more clearly ascertained, some embodiments will now be described, by way of example, with reference to the accompanying drawings, in which:

FIGS. 1A and 1B are example block diagrams of systems for a respiratory condition treatment architecture, in accordance with some embodiment;

FIG. 2A is an example block diagram for a first operation of the therapy system, in accordance with some embodiment;

FIG. 2B is an example block diagram for a first operation of the therapy system with emergency response, in accordance with some embodiment;

FIG. 2C is an example block diagram for a second operation of the therapy system, in accordance with some embodiment;

FIG. 2D is an example block diagram for a second operation of the therapy system with emergency response, in accordance with some embodiment;

FIG. 3A is an example block diagram for the analytics server, in accordance with some embodiment;

FIG. 3B is an example block diagram for the emergency services server, in accordance with some embodiment;

FIG. 4 is an example block diagram for the analytics module, in accordance with some embodiment;

FIG. 5 is an example block diagram for the recommendation module, in accordance with some embodiment;

FIG. 6A is an example illustration of a patient interacting with a sensory device, in accordance with some embodiment;

FIG. 6B is an example illustration of a sensory device screen output, in accordance with some embodiment;

FIG. 7 provides a flow diagram for an example process of respiratory condition therapy management, in accordance with some embodiments;

FIG. 8 provides a flow diagram for data analytics for the respiratory condition therapy, in accordance with some embodiments;

FIG. 9 provides a flow diagram for an example process of collection of additional physiological data, in accordance with some embodiments;

FIG. 10 provides a flow diagram for an example process of emergency response during a respiratory therapy session, in accordance with some embodiments;

FIG. 11 provides a flow diagram for an example process of location confirmation, in accordance with some embodiments;

FIG. 12 provides a flow diagram for an example process of patient monitoring, in accordance with some embodiments;

FIG. 13 provides a flow diagram for an example process of the emergency response workflow, in accordance with some embodiments;

FIG. 14 provides a flow diagram for an example process of patient triage, in accordance with some embodiments;

FIG. 15 provides an example illustration of a screenshot of a healthcare worker’s interface, in accordance with some embodiments;

FIG. 16 provides an example illustration of a screenshot of a patient’s device login interface, in accordance with some embodiments;

FIG. 17 provides an example illustration of a screenshot of a patient’s device staging interface, in accordance with some embodiments;

FIG. 18 provides an example illustration of a screenshot of a patient’s device runtime interface, in accordance with some embodiments; and

FIGS. 19A and 19B provide illustrations of possible computing devices capable of performing the above mentioned respiratory condition therapy, in accordance with some embodiments.

### DETAILED DESCRIPTION

The present invention will now be described in detail with reference to several embodiments thereof as illustrated in the accompanying drawings. In the following description, numerous specific details are set forth in order to provide a thorough understanding of embodiments of the present invention. It will be apparent, however, to one skilled in the art, that embodiments may be practiced without some or all of these specific details. In other instances, well known process steps and/or structures have not been described in detail in order to not unnecessarily obscure the present invention. The features and advantages of embodiments may be better understood with reference to the drawings and discussions that follow.

The present invention relates to systems and methods for the improvement of therapies for respiratory conditions, such as chronic obstructive pulmonary disease (COPD). In particular, the present respiratory condition therapy system allows for remote access by an agent (virtual or otherwise) to manage respiratory condition exercise therapies. It should be noted, however, that other pathologies that rely upon exercise therapies may benefit from the systems and methods disclosed herein; as such the following systems and methods may apply to situations where these other pathologies are being treated (e.g., obesity, cardiac pathologies, etc.) with minimal alteration in the operation of the system. Thus, while this disclosure may center upon the treatment of respiratory conditions, it is not intended to limit the scope of this disclosure or the functioning of the instant systems and methods. The present invention also relates to systems and methods for the providing of emergency responses during such therapies.

Further, it should be noted that the sets of embodiments detailed in this disclosure are for illustrative purposes only and are not intended to artificially limit the scope of the invention. As such, terms such as “must”, “can’t” “will” and other such limiting language, is intended to only apply to the one instant embodiment being contemplated. Such restrictions are not intended to expand to other embodiments which may have a scope significantly broader than the instant substantiation.

Additionally, within this disclosure, one will notice that for particular parts and entities, more than one naming convention may be applied. This is not intended to differentiate between these elements, but merely is stylistic choices in wording. For example, a “user” and a “patient” are meant to be synonymous as the individual who receives therapies. The terms “agent” or “therapist” is used extensively herein, however this term is intended to include any sort of healthcare worker. For example, the patient could be working with a physical therapist, registered nurse, doctor, physician’s assistant, or other healthcare worker, and this would all be considered under the term “therapist”. The server may be referred to as an “analytics server” or a “backend server” depending upon context. The database may be a “data store”, “memory”, or merely “data”. Again, these choices of wording do not, in any way, artificially limit the scope of this instant disclosure.

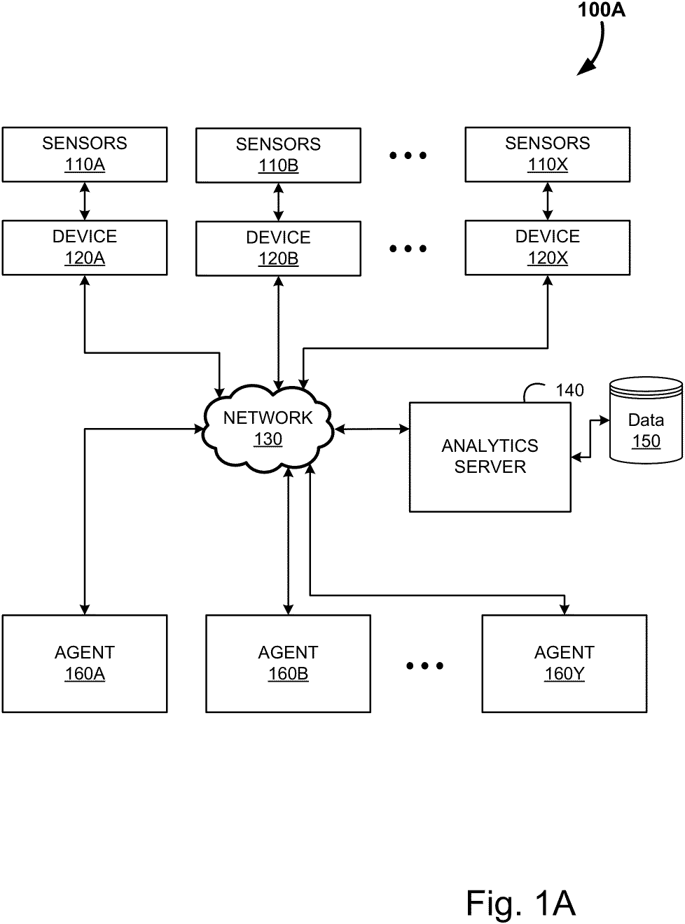

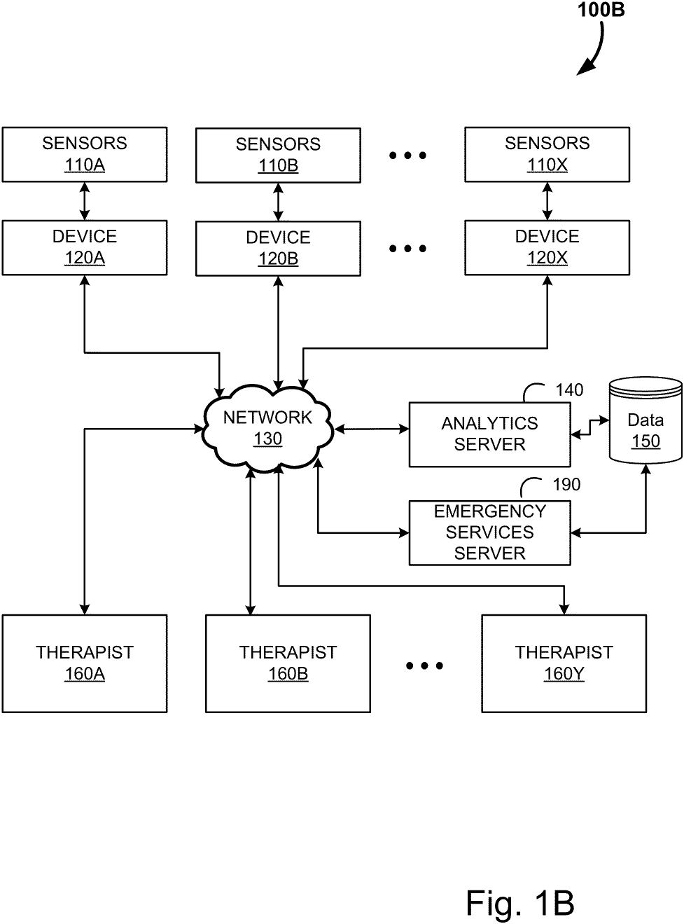

In order to facilitate the discussion, FIG. 1A provides an example environment where the remote treatment of respiratory conditions (or other pathology) is performed, shown generally at 100. In this example, one or more users (not illustrated) interact with one or more sensors 110a-x. The sensors include, at a minimum, an optical sensor for collecting heartrate and blood oxygenation levels, and blood volume changes in the location being monitored. Oxygen saturation values may alternatively be collected by an infrared sensor or the like rather than a wearable device, in some embodiments. Additional sensors may include audio/video sensory inputs, blood pressure sensors, temperature sensors, electrocardiogram (ECG) signals, etc. In some embodiments the sensors may be housed in a wrist mounted device (with or without displays provided), with an attached optical sensor. In some embodiments, the optical sensor is coupled to the wrist mounted device, and includes a finger sleeve for sensing optical signals. In other embodiments, ECG signals and the like may be collected by leads connected to the patient’s chest. Video and audio data collection may be performed by the patient’s device 120a-x, as these devices typically also have integrated microphones and cameras. In alternate embodiments, when such audio and video are being collected, the data may be procured using some external video device (e.g., a webcam or even an integrated camera in the wrist mounted device).

The sensors couple to a user device 120a-x. The user device 120a-x may include a capable computerized device, including a desktop computer system, a laptop computer, a tablet, a smartphone, or a smartwatch. The only requirements for the user device 120a-x are that it includes a screen interface, an audio interface, and a network connection.

One or more agents 160a-y are also connected to a device (not illustrated). As previously noted, the agent may be a respiratory therapist, a physical therapist, a coach, a clinician, trainer, or similar individual. Again, the agent device may include a desktop computer system, a laptop computer, a tablet, or a smartphone. The only requirements for the agent’s device are that it also includes a screen interface, an audio interface, and a network connection. The agent’s device also connects via the network to share video and audio information with the user’s device 120a-x. This may be accomplished by Zoom® or a similar video conferencing platform. Alternatively, the system may include a custom-built video sharing application. In addition to the video conferencing connection between the agent 160a-y and the patient, the sensor data is also transmitted between the parties.

In some embodiments, the number of agents 160a-y are one-to-one with the number of patients. In alternate embodiments, it is possible for a single agent 160a-y to service many patients at the same time. Likewise, one (or more) patients may be serviced by a number of agents. This one-to-many approach, when scaled, is untenable in traditional therapy settings (at least efficaciously), but with advanced notifications regarding the patients’ wellbeing (calculated by the analytics server 140, and/or device(s) 120 and 230) such one-to-many, at scale, handling of patients by a single agent is made possible.

Additional data, including the sensory information, may also be displayed on the user’s device 120a-x, and this data may be transferred to the agent 160a-y via the network. This sensory data is also provided to an analytics server 140 for storage in a data store 150, and for processing to generate analytics, alerts and notifications. The data store may include any known data storage architecture. In some embodiments, structured query language (SQL) may be employed to manage the data store 150. Additionally, control signals may be output by the analytics server 140. These outputs may be shared with the healthcare worker and/or the patient.

In some particular embodiments, the analytics server may include a virtual agent capability that can either augment the agents 160a-y, or in some cases entirely replace the need for a human agent entirely. This virtual agent may include an avatar and provides the patient instructions in the same manner a ‘real’ agent 160a-y would. Details of the operation of such a virtual agent will be provided in greater detail below.

The information between the user’s device 120a-x, the agents 160a-y, and the analytics server 140 is transmitted over a network 130. The network may include any known network types, such as a local area network (LAN), a wide area network, a wireless LAN (WLAN), a cellular network, an internal corporate network, or some combination thereof. In some embodiments, the network 130 may include the Internet for transmission of data between the agents 160a-y, the server 140, and the users’ devices 120a-x.

Turning to FIG. 1B, a similar architecture is illustrated with the exception of an additional module. This module is an emergency services server 190. Likewise, sensory data is also provided to an emergency services server 190 for reaction when an emergency (or intervention) event is detected. The emergency services server 190 may also access the data store 150 for patient history, location, and sensory data. In some embodiments, the network 130 may couple each of the agents 160a-y, the server 140, the emergency services server and the users’ devices 120a-x together. It should be noted that, in some embodiments, the analytics server 140 and the emergency services server 190 may be co-located in a backend health services control center (not illustrated). However, due to the functional differences between these modules, they may be illustrated separately in reference to this and following figures for the sake of clarity.

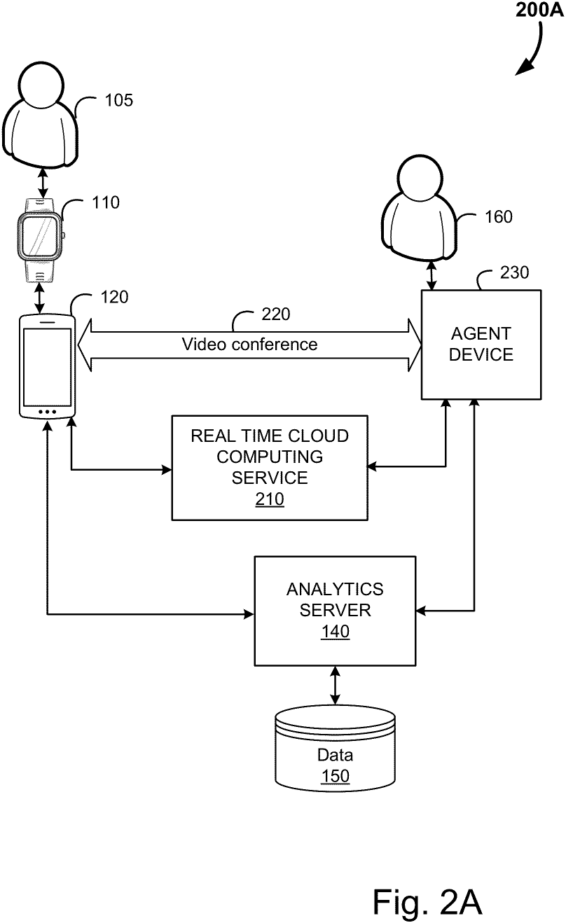

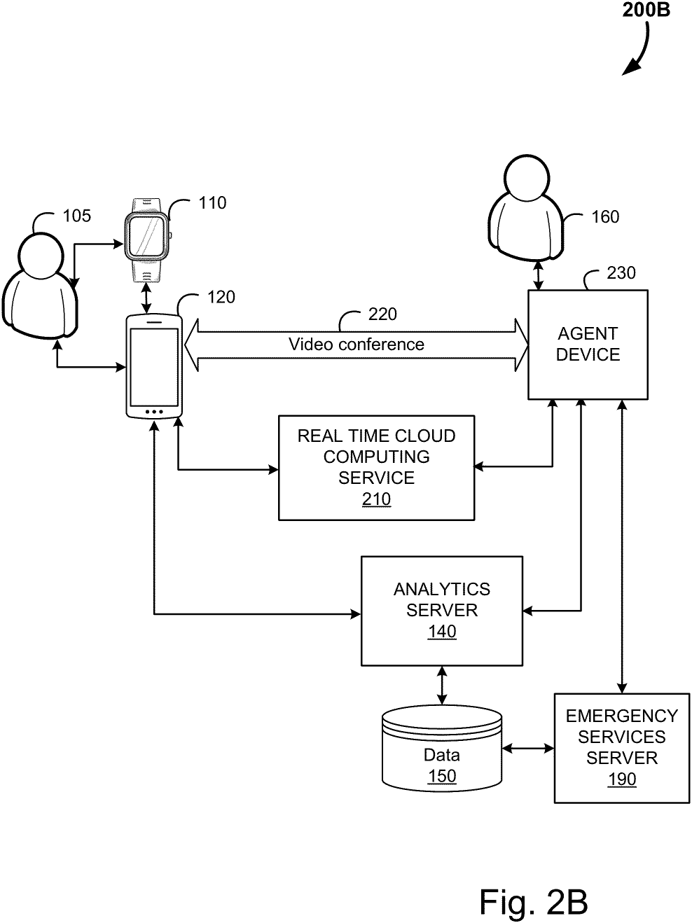

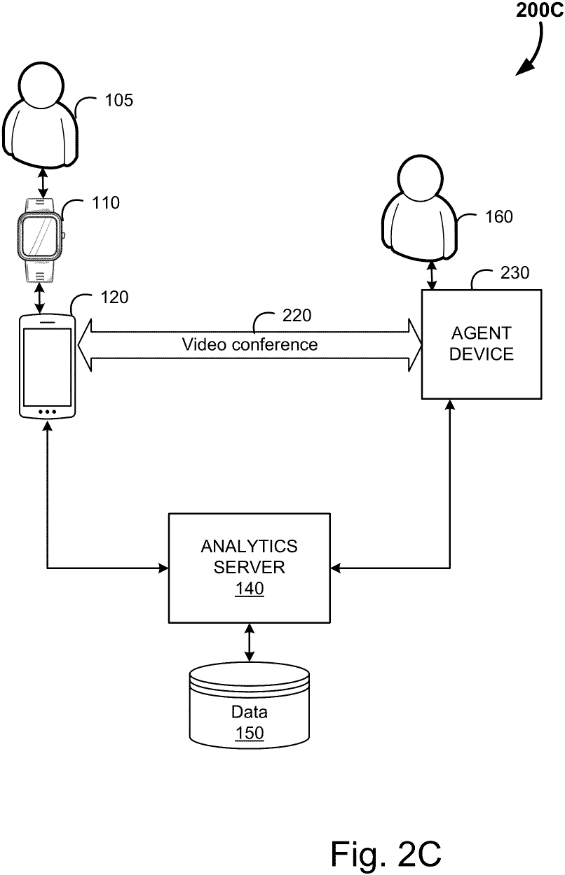

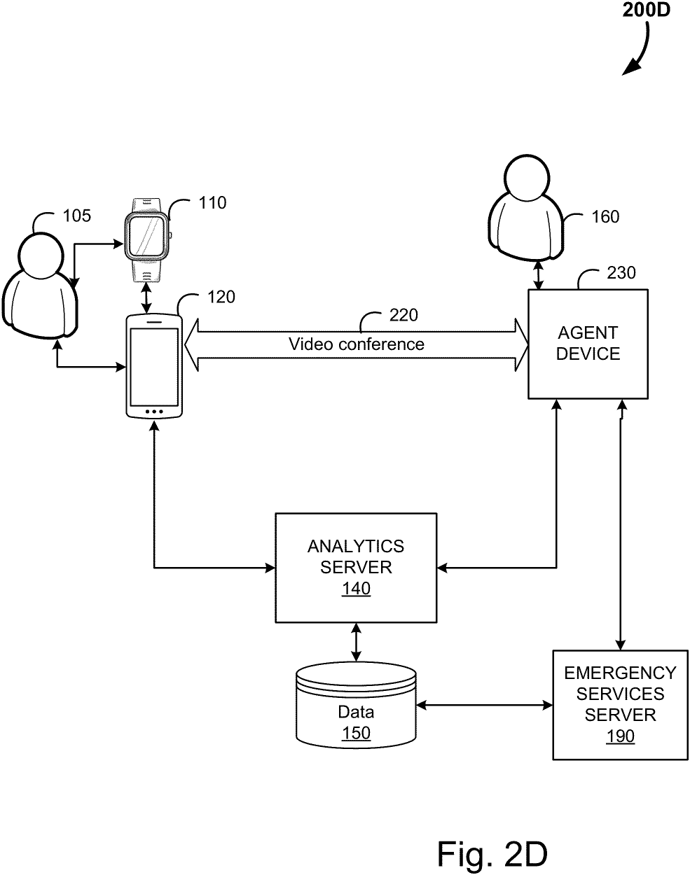

FIG. 2A provides an illustrative block diagram for a first example of the functional communications between the various components of the example system, shown generally at 200A. In this example, there are three separate communication channels in play between the user 105, and the healthcare worker. The first, as discussed, is a video conferencing channel 220. This occurs between the patient’s device 120 and the agent’s device 230.

The patient 105 has their various physiological data collected by the sensor(s) 110, and in the case of audio and video signals, via the device 120 itself. The sensory data may be transmitted two distinct pathways, in this instant embodiment. One path provides at least some portion of the collected data directly to the agent’s device 230 (generally a laptop, smart phone, tablet, desktop, or similar device) via a real-time cloud computing service 210. In some embodiments, this computing service 210 may include Microsoft Azure, AWS, or a similar infrastructure.

In some embodiments, the information transferred over the computing service 210 includes the physiological data that has been collected, such as heartrate, blood oxygen saturation, and calculated Photoplethysmography (PPG). PPG calculations may be performed directly in the sensory device 110, on the user’s device 120, or on the agent’s device 230, due to the low computational resources needed to make said calculations. In some embodiments, ECG and other data may likewise be displayed.

Sensory data may also be transferred to the analytics server 140 via the database, for more in depth analysis and computations. The output of these analytics may be sent to the agent’s device 230 as well. For example, video and audio signals from the patient’s device 120 and/or the sensor(s) 110 may be analyzed (by a rule-based waveform pattern analysis or machine learning algorithm) to determine breathing rates, estimated breath volumes, and the like. Such real-time analysis is too computationally intensive to be performed by the devices 120 and 230, let alone the sensory systems 110. Such computed outputs by the analytics server 140 are then provided to the agent’s device (and possibly the patient’s device 120).

The devices 230 are thus able to generate and/or display notifications or alerts to the agent/healthcare worker 160 regarding the health and wellbeing of the patient 105. For example, if the patient’s breathing or heartrate enter dangerous territory, the healthcare worker may be notified as such, and the activity level adjusted accordingly. This allows for two things: 1) safer exercising situations for the patient, and 2) allows the healthcare worker to not need to have their full attention on a single patient. This allows for on-to-many treatments of patients by the healthcare worker. This is more efficient and can drop costs for the patients considerably.

In addition to calculating breathing rates, for example, many analyses may be performed by the analytics server 140. The following list of possible analyses is non-inclusive. Further, none of the following analytics need be performed. For example, trends in patient health may be calculated by the server, but the breathing rate not calculated. In another embodiment, both metrics are calculated and provided to the agent/healthcare worker 160 (possibly one in real-time and the other as a preview before therapy starts). Data from the data store 150 is leveraged in any given analysis, and used to store analytics as they are developed.

Different analysis may include, by way of example, breathing rates and/or volumes. Trends in physiological measurements (e.g., blood pressure), trends in general health (e.g., trends in heartrate for given activity levels over time), and a “symptom score” which may be a collated metric that combines the trends in health of the patient, with physiological data to estimate either specific symptoms/pathologies for the patient or may indicate general health of the patient. The “Symptom score” may include validated questionnaires (e.g., COPD Assessment Test (CAT), or other symptoms such as fatigue, breathlessness, activity, and cough, etc.) combined with collected physiological data, over a longitudinal period.

In addition, long term trends of pulse and oxygen saturation for the patient may be captured and stored for display to the patient directly. This may be a motivational element/gamification technique to improve patient compliance with therapy routines. Other feedback that may be provided to the patient includes letting the patient know if the exercise intensity was appropriate, making recommendations for the next session, providing recommendations for exercise frequency, and supplemental exercises outside of a healthcare worker driven session. The long-term metrics/health of the patient, and the trends in these patterns may be of particular use when designing an exercise routine for the patient. For example, a patient who is getting worse, or stagnating in their progress may have a recommendation for more frequent, lower intensity at home/non-supervised exercises, while a patient that is already improving may not require these supplemental routines. The collected data is designed to teach the patient and keep them engaged in their therapy. Additionally, the healthcare worker can directly communicate with the patient outside of sessions (e.g., in-app messaging, external email, phone or video calls, etc.) to provide feedback or motivational communications.

Data analytics may additionally be provided to the healthcare worker and/or a physician (or other clinician) for review. This can help the physician understand the long-term changes in the patient’s health, how they respond to alterations in their exercise routines, and how supplemental oxygen may impact their activity. Data tracked includes not only the collected physiological data, but also adherence to the therapy routines, external activity levels, diet, and other metrics. Individuals with a lower compliance to the therapy routines may be designated as “high touch” and may receive more personalized attention in order to improve their motivation. This personalized attention may go as far as communications (e.g., emails, phone calls, texts, etc.) and even in-person contact.

Turning to FIG. 2B, a very similar architecture is provided, shown generally at 200B. However, in this example architecture, the agents device 230 and the database 150 additionally couple to the emergency services server 190. Emergency analysis and response information may be generated by the emergency services server 190 and provided independently to the agent’s device (and possibly the patient’s device 120), as well as optionally a third-party medical intermediary, and emergency medical services (EMS). EMS may include traditional 911 dispatcher services, direct hospital and/or ambulatory connection, police or fire departments, and the like. Generally, however, the term EMS is primarily dedicated to emergency response dispatcher systems (although not limiting).

FIG. 2C provides another example architecture of the functional communications between the various components of the example system, shown generally at 200C. In this example, the cloud service is omitted, and all sensory data, as well as the computed data, is provided between the patient’s device 120 to the agent’s device 230 via the analytics server 140 directly. This arrangement requires that the analytics server 140 have significantly more computational resources compared to the prior model. However, the presently disclosed system has some added advantage in the protection of personal protected information (PI) handling over the prior model. Similarly FIG. 2D provides the same basic infrastructure with direct communication through the analytics server 140, with the emergency services server 190 in communication with the agent’s device 230 via the analytics server 140 (via the database 150).

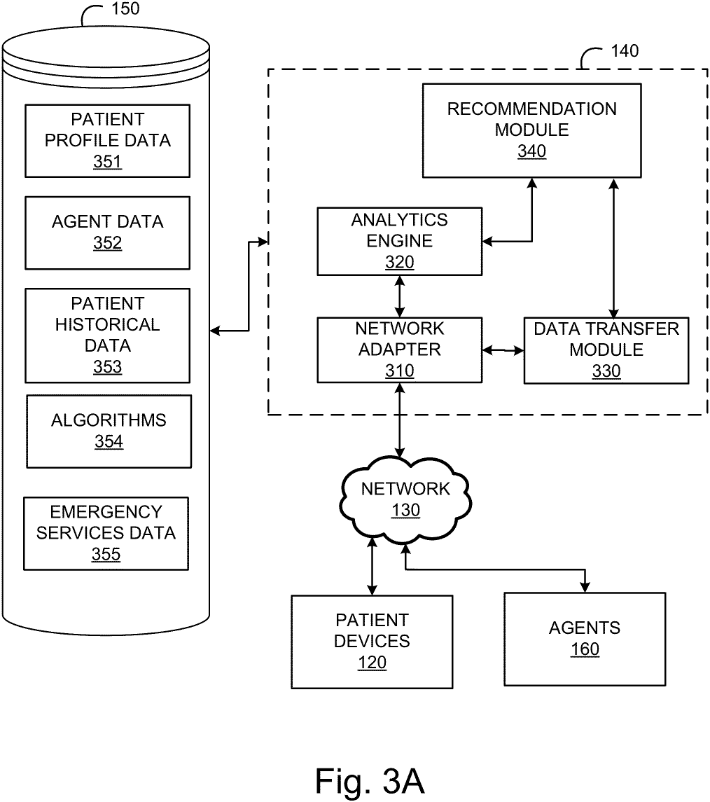

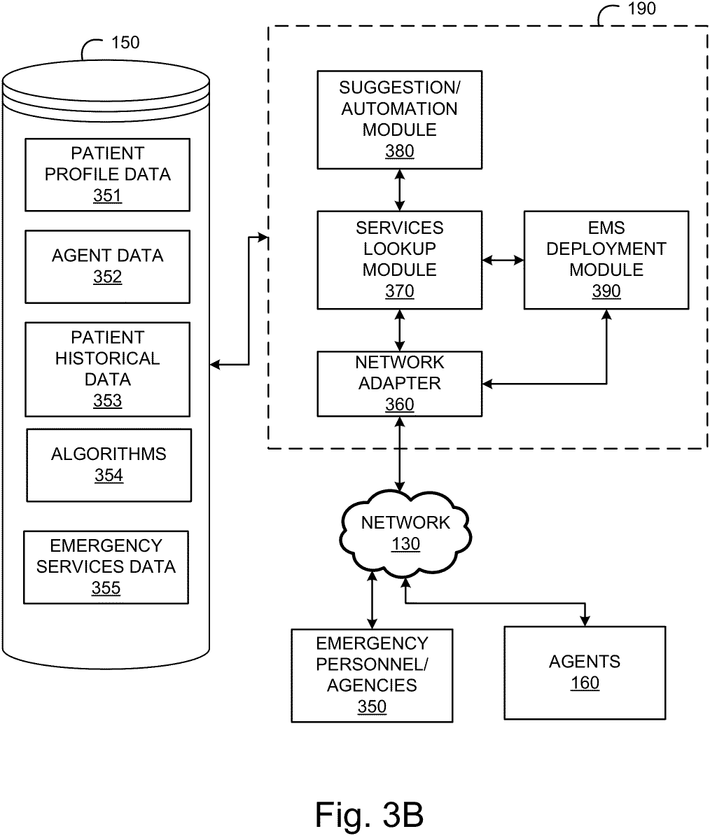

Turning to FIG. 3A, a more detailed description of the analytics server 140 and the data store 150 are provided. The data store includes details regarding the patient’s profile 351, the agent 352, patent historical data 353 (for calculating trends) and algorithms used in analysis 354. These algorithms may include waveform fitting type algorithms (to identify when the PPG signal indicates accurate pulse and oxygenation readings, for example), rule-based calculations for notification generation, or more complex machine learning (ML) algorithms for determining health trends, possible pathologies, and the generation of a symptom score.

In some embodiments the ML models/algorithms are deep neural network algorithms. The AI models may be predictive for individuals that may benefit the most from respiratory condition rehabilitation and exercise therapy. Further, the degree of attention required for a patient may be discovered using these algorithms. Such “high-touch” patients may require additional monitoring by a human agent or clinician due to fragility and/or increased risk of complications that may have otherwise gone undetected. Importantly, the AI models may also predict the risk of respiratory condition exasperation in a given time period (e.g., one year out from the prediction date). This risk analysis may be based upon collected data described herein, and other payer supplied data. This prediction may then be compared with a clinician prediction of outcomes and used to guide future monitoring and/or treatment of the individual.

All of these predictive analytics may be leveraged to assist in the care of the individual by supplying guidance on when to onboard a patient, and the frequency that a patient should be in contact with a healthcare worker in order to successfully have them start pulmonary rehabilitation. Other guidance that may be supplied by the models may include the automation of a series of tests (e.g., 6MWT, sit-to-stand, box test and shuffle test, etc.) in conjunction with oxygen monitoring. This system may notify the physical trainer if there is a need to reduce, increase or stop the testing, as discussed in greater detail herein. The system may also automate balance testing in the home environment.

Patient and healthcare worker profile information may be provided to the other party to “humanize” the other individual. Having context for the patient’s condition also has the added benefit of allowing treatments to be tailored to the individual. For example, if the patient’s profile indicates the patient has an injured knee, exercises may focus on upper body activities, and resistance bands may be employed for lower body exercises over any activities that involve shock to the knees. Lastly, in some embodiments, the profile information may be leveraged to match the patient with the healthcare worker, in some cases. For example, a patient may have preferences as to the age or gender of their healthcare worker. Likewise, the healthcare worker may prefer working with individuals with certain forms of respiratory conditions, and/or individuals operating at a specific activity level. Having the profile information may allow these matches to occur.

Also, the data store 150 may include information regarding personality and code behind a virtual agent (not illustrated). A virtual agent may leverage the models to provide coaching without the need for a human agent to be present. Rule-based alerts and notifications may be converted into therapy commands by the virtual agent program. An avatar may be employed to make the virtual agent more palatable to the patient.

The analytics engine itself includes a network adapter 310 for coupling to the patient device 120 and the agents 160, via the network. The analytics engine 320, uses the collected data received via the network adapter 310, as well as information already contained in the data store 150, leveraging the models/algorithms 354, to generate the notifications, and other analyses already described herein.

A recommendation module 340 may utilize the analytics, using Boolean rule-based analysis, to generate recommendations. In other embodiments, statistical machine interface, neural network machine learning, and/or deep neural networks may be leveraged to generate recommendations. For example, if the analytics engine determines that, based upon historical data a given individual’s maximum heartrate shouldn’t exceed 145 (compared to the average of 150 beats per minute for a typical 65-year-old), the recommendation module may suggest a decrease of activity when the patients heartrate has reached 135 beats per minute (bpm) based upon the speed of the increase of the heartrate trajectory at a given effort level. In another example, the same system may recommend a different degree of activity adjustment only at 137 bpm if the heart rate has only marginally increased over time at the given activity level.

A data transfer module 330 provides the notifications and recommendations (and in some embodiments emergency alerts) back to the network adapter 310 for output. The network adapter may ensure that the proper data is forwarded to the proper agent 160, patient, or given authority. In some embodiments, the system may also forward data to other interested third parties (the patient’s primary doctor, for example).

FIG. 3B provides a more detailed illustration of the emergency services server 190. This system also leverages many of the same data sources as the analytics server 140 with the addition of leveraging an emergency services data 355 store. This datastore includes contact information for a variety of emergency personnel and/or agencies 350.

The emergency services server 190 has a network adapter 360 for connecting to the network 130 for connectivity to the agents 160, the emergency services personnel and agencies 350 and, in some embodiments, third-party triage and escalation medical services (not illustrated). One possible element of the emergency services module 190 is to confirm the physical location of the patient(s) and match these locations to the appropriate emergency personnel or agencies 350. This enables extremely rapid connectivity between the patient, agent, and third-party medical provider when required. Alternatively, it also allows the healthcare worker to call a direct EMS phone number for the location of the patient, or use ‘rapid connectivity’ through an automated system.

A suggestion/automation module 380 may take information collected from the patient and generate automated emergency response actions (in some cases), or at a minimum, generate suggestions regarding interventions and emergency contact. This system may notify the physical trainer if there is a need to reduce, increase or stop the testing. The system may also automate the balance of testing in the home environment. In other embodiments, as noted the virtual agent may perform the bulk of the therapy, but if the sensors pick up anything too far out of the norm (a configured range for each of the sensor data- e.g., a pulse rate above 150 for a 65-year-old, by way of example), then the suggestion/automation module 360 may pull in a human agent to assist with the issue at hand. In this manner, a single healthcare worker could conceivably render therapy to a relatively large cohort of patients simultaneously (e.g., 10-30 patients at a time).

In addition to providing notifications and alerts to the healthcare worker when ranges of measured physiological data are outside of normal bounds (as configured by the healthcare worker, doctor, or pre-set by specialists), the suggestion/automation module 360 may also be configured to generate emergency responses, including alerting medical staff/ambulance if certain conditions are met. For example, if ECG samples are being collected, and the signals indicate the patient is suffering from a heart attack, the system may automatically call the paramedics. Known signal analysis techniques may be leveraged to identify if an individual is experiencing a heart attack, or ML analysis may likewise be employed. Likewise, ML models that analyze audio and video data may also alert the authorities. For example, an individual calling for help, or a person falling/fainting, may trigger an emergency response. Likewise, oxygenation and/or pulse measurements may trigger an emergency response if found within dangerous ranges. In addition to the system generating alerts, the patient themselves may be able to initiate an emergency response. This may be through a voice activated system, such as the patient saying, “I need help” or similar utterance, or through the patient selecting a built-in alert ‘button’. Such a built-in emergency response button would enable the user to quickly activate an alert and may also provide the patient the ability to “de-select” the alert in case it was initiated inadvertently.

In addition to merely contacting emergency personnel of the emergency, the system may provide additional data to the first responders that will assist in the delivery of care via the EMS deployment module 390. This may include providing the address of the individual, and in cases where the patient’s device includes GPS, exact coordinates of the patient. In the event the patient lives in a multi-dwelling building, the system may request the patient supplies information relevant to getting to their specific domicile. These details may be requested in advance or in real-time upon connectivity of the audio and video feeds. Additional data, such as collected vitals (e.g., blood pressure, temperature, pulse and blood oxygenation levels) may likewise be provided. Other individuals nearby, and even live visual and audio feeds may even be provided.

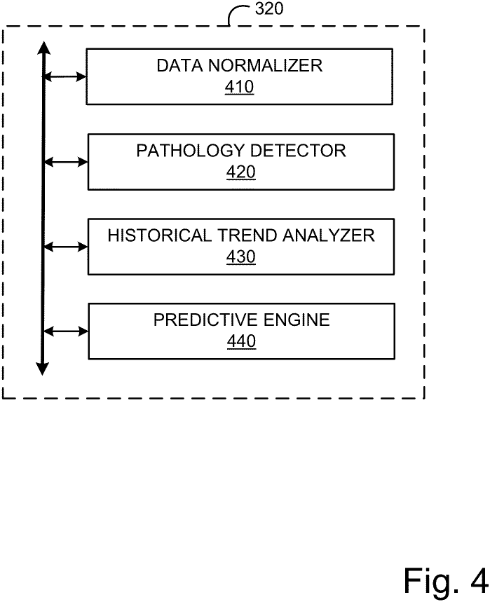

Turning now to FIG. 4, a more detailed block diagram is provided for the analytics engine 320. The first module of the analytics engine 320 is a data normalizer 410, which can receive the raw data and perform cleaning and adjustments as is necessary. Based upon the photoplethysmography (PPG) signals, for example, some of the raw data may be discarded, or flagged as unreliable. In some embodiments the photoplethysmography signal includes a waveform characteristic. This waveform may be compared with a desired wave shape, via any known graph matching algorithms. In some embodiments, the shape of the recorded PPG is compared against a “saw-toothed” shape via a least means squared or similar technique. In some embodiments, the peaks and valleys of the prototypical control waveform are aligned to the recorded waveform. The peak heights are then normalized, and the least means squared calculation performed.

Signals that fit the desired saw-tooth shaped curve within a certain threshold are indicative that the other measurements of pulse and oxygenation level of the blood are accurate. PPG signals that are not in a saw-tooth shape indicate these two signal types are not to be trusted (the accuracy is in question). In some embodiment, based upon how closely the PPG signal shape matches the prototypical saw-toothed shape, the degree of accuracy of the oxygenation percentage and pulse rate may be estimated.

In some embodiments, the data normalizer 410 may discard data inputs that have an accuracy below a configurable threshold, or conversely signals where the PPG shape deviates by a configured threshold from an anticipated saw-toothed shape. In some other embodiments, this data is retained as it may have some value for machine learning (ML) algorithms, but is flagged as lacking accuracy. The terms “lacking accuracy”, “unknown accuracy” or “possibly inaccurate” may all be utilized rather than “inaccurate” because the recorded signals may indeed be accurate; however, this cannot be counted upon if the PPG signal lacks the saw-toothed wave shape.

In addition to cleansing the incoming data in this manner, a series of transforms may be applied to further normalize the raw data. For example, different sensor manufacturers may have sensors that measure the same physiological information, but may routinely yield slightly varying outputs. For example, a GE blood oxygen sensor may measure a 0.02% lower oxygen saturation measurement as compared to a sensor manufactured by Medtronic. While such a difference in measurements may be inconsequential for a single session, when performing analysis over a longer time period it may be relevant. Transforms thus may include adjustments by sensor types/manufacturers, time of day, ambient temperature, trainer, patient’s condition/age/weight/etc., and the like.

The data, one normalized, may be provided to a pathology detection module 420 for analysis by rule driven algorithms or via ML models. For example, in the case of a rule driven analysis, a comparison of the patient’s age, blood oxygen saturation, and weight may be factored together to identify if the patient has congenital heart failure. In another embodiment, the patient’s blood pressure, activity levels, weight and age may be utilized to determine if the patient suffers from hypertension. Certain ECG signals may indicate a heart palpitation or even a heart attack. The pathology detector 420 may also consume very large data sets in order to identify pathologies using ML models. These models may include neural network AI models. In some embodiments, thousands of patient datasets with known pathologies are fed into the model to train it. The trained model may then be employed to identify similar pathologies in the newly collected datasets.

In some embodiments, the information used for identifying a pathology is not a single picture of the patient’s condition, but rather a time domain progression of data. For example, a shift in the blood oxygenation of the patient over time may indicate cardiac functioning issues. The historical trend analyzer 430 may be employed to identify these changes over time, and when present feed the trends into the rule based models or the ML models to identify any pathologies, and maybe more importantly, allow the predictive engine 440 to extrapolate from the collected data to generate expected future data. These predictive models may again be simple, such as continuing the trend as a linear function while applying a decaying element. In other embodiments, ML models may again be employed to predict future data for the patient. By predicting the future condition of the patient, pathologies that have yet to occur may be proactively intercepted and/or changes may be employed to improve the outcome of the patient. Machine learning algorithms may also extrapolate timeseries graphs and predict arbitrary outcomes that are correlated with the available data. These models may also explain/make intelligible the reasons why a particular prediction was made (e.g., 75% of the reason the model predicted exacerbated a respiratory condition is due to the levels of SpO2 measured in the past 72 hours).

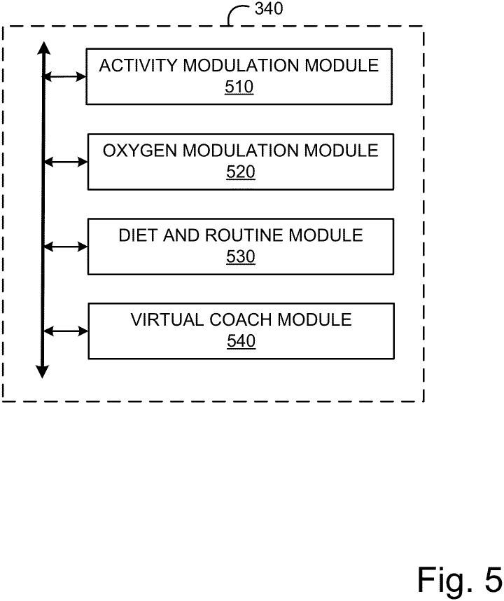

Turning now to FIG. 5, greater detail of the recommendation engine 340 is provided. This recommendation module may provide activity level suggestions to the agent 160 at a minimum or may go as far as to provide an entirely automated therapy session. In some embodiments, the activity levels of the patient may be modified/notified by the activity modulation module 510. This module may employ rules to determine when the activity should be increased or decreased based upon pulse rates and oxygen saturation levels (and possibly temperature, blood pressure and/or ECG inputs). These adjustments may take into consideration a desired range for these various data points, and the trends of the signals. For example, a patient of a specific age may have a target heartrate of 135-145 beats per minute (bpm). The individual may only be at 123 bpm, but has been increasing at a rate of 4 bpm every minute. In this instance the system may recommend continuing current activity levels. A similar situation, where the patient is only increasing at a rate of 2 bpm per minute may have a suggestion of a moderate increase in activity levels.

In some embodiments, the values of differing physiological data for the patient may be at odds with one another. For example, an individual may have extremely high blood pressure even though the pulse rate is below a given target. In these embodiments, the values may be reconciled to make a determination on how to adjust activity levels. In some cases, this is done by comparing the measured physiology against the target level, and applying a weight to the difference. The weight may be a constant based upon the physiological data type. For example, pulse may be given a greater weight than blood oxygen saturation in some particular embodiment. In alternate embodiments, the weights are not constants, but rather increase exponentially based upon the degree a measured value is off from the target. In this example, being above a target pulse rate by a couple of beats per minute may be given a relatively low weights, whereas being 5 bpm over the target may be a much larger weight. Alternatively, the weights may be complex functions designed to avoid any dangerous conditions (e.g., extremely large weights given to the pulse rate once it is above a dangerous threshold).

In addition to recommending a change in activity, the recommendation engine 340 may include an oxygen modulation module 520 for patients who are receiving supplemental oxygen. This module may rely primarily upon blood oxygen saturation to suggest increasing or decreasing the supplemental oxygen ratios. This generally is a simple function (in some case linear, and in others exponential) comparing the oxygen saturation against a target, and the difference being used to calculate a modulation in the supplemental oxygen settings.

A diet and routine module 530 may employ trends in the patient’s measured physiological data, in addition to profile data to provide recommendations regarding dietary intake, supplemental exercise activity, water consumption, and the like. Again, rule based functions that consider time domain trends may be of particular use in generating these recommendations.

Collectively, these recommendations may be output directly to the patient and/or the trainer. In some embodiments, only the trainer is provided these recommendations, and the trainer will curate the recommendations before supplying them to the patient. On the other extreme, the system may employ a virtual coach module 540 to provide the patient these recommendations/instructions directly via a specialized interface. The interface may, in some cases, include a virtual avatar. In some particular embodiments, when the recommendations are clear/within a given confidence level, the system may provide the instructions to the patient directly, but when there is ambiguity present/there is a low confidence level, the system may provide a trainer with the recommendation data and request their intervention. This hybrid approach may allow a single trainer to simultaneously interact with a number of patients, thereby increasing therapy efficiency significantly.

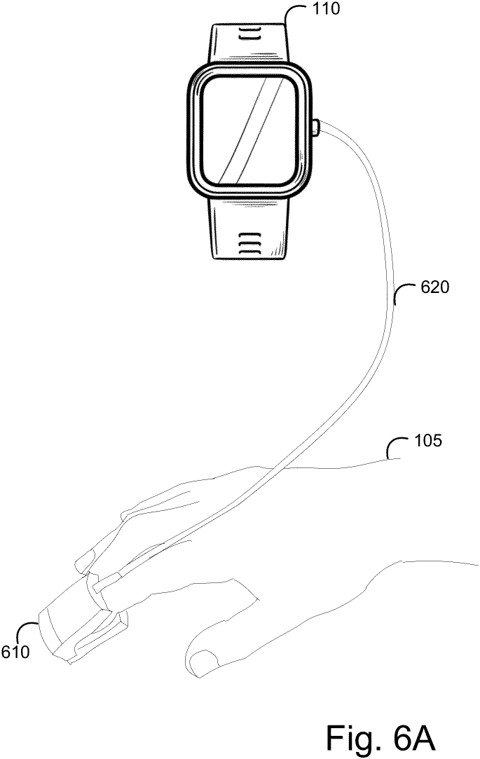

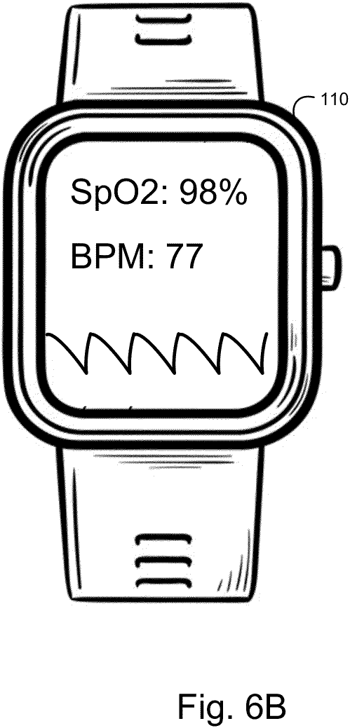

Turning now to FIG. 6A, an example of a patient interfacing with one set of sensors is provided. In this embodiment the patient 105 is seen with their finger placed within a sensor 610. This given sensor include an optical sensor for measuring pulse, PPG and blood oxygen saturation. The optical sensor 610 couples either by wire 620 or wirelessly (e.g., Bluetooth or similar protocol) to the sensor device 110. Here the sensor device 110 is displayed as a wrist mounted device. It should be noted that this sensor arrangement is but a single embodiment, and alternate systems that employ chest sensors for ECG measurements may be employed. In yet other cases, different sensor embodiments are possible. For example, in some particular instances, rather than a finger sensor and a wrist mounted device, the sensors may be incorporated into a headband, ear worn device, armband, or other body worn device.

The finger sensor 610 may likewise include optical sensors for blood pressure measurements, temperature sensors and the like. In some embodiments, the wrist mounted device110 (or similar wearable device) may collect the body temperature and blood pressure data. In some embodiments, one or more of the sensory components may include accelerometers and/or gyroscopes for sensing patient movement. This movement data may be employed to estimate the degree of patient activity, and/or during data normalization. For example, a swinging of the arm may induce a centrifugal force that may distort a blood pressure measurement. By knowing the direction and speed of the movement, the blood pressure measurement may be corrected accordingly. The same goes for pulse rates that may be impacted by movements/jarring, and other collected physiological information.

FIG. 6B provides an example view of the wrist mounted device110 (or other device) interface. At a minimum, this device may provide the user with a blood oxygen saturation value, pulse rate, and the PPG waveform. Note the present image includes a “good” PPG waveform that resembles a “saw-tooth” shape.

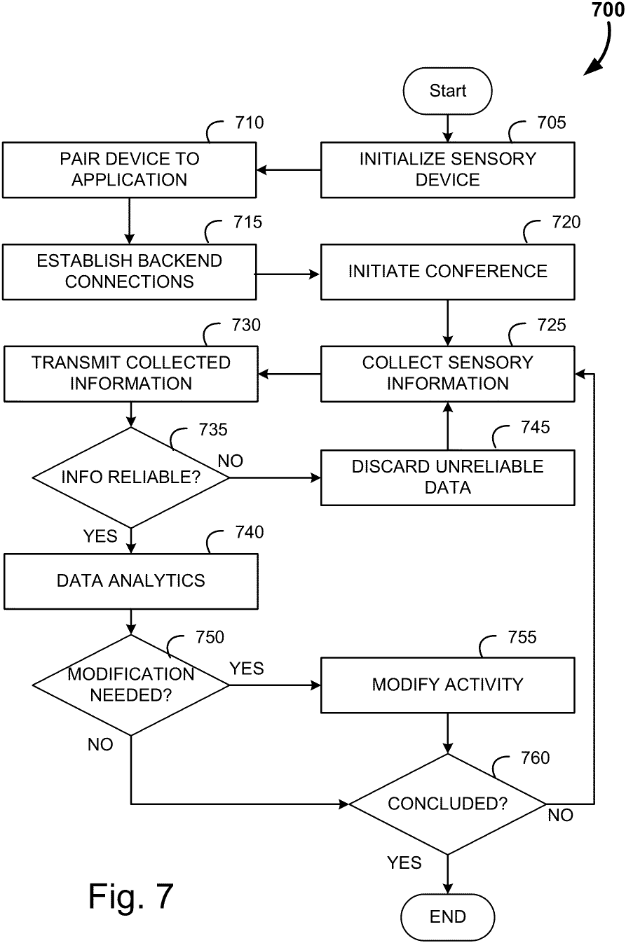

Moving on, FIG. 7 provides an example flow diagram of the therapy improvements, shown generally at 700. In this example process, the sensory device is connected to the patient and initialized (at 705). Initialization includes turning the device(s) on and collection of physiological data from the patient. The device is then paired (at 710) to the application operating on the patient’s device. This pairing may include a wireless connection (e.g., Bluetooth), or via a wired solution. Regardless, once the sensor(s) are paired to the application, the application establishes one or more backend connections (at 715) to, at a minimum, the backend server, and in other embodiments, via a cloud computing service with the agent’s device.

A conference (usually a video conference) is then established between the patient and the healthcare worker (at 720). In some particular embodiments, the video conference is established with a virtual agent generated by the backend server. In other embodiments, the video conference is between a virtual coach as well as a human healthcare worker, allowing for intervention by the human healthcare worker when needed. In some embodiments, the conference is over a known and commercially available service (e.g., Zoom, Google Hangouts, Microsoft Teams, etc.). In other embodiments, the conference is a HIPAA compliant proprietary channel via the backend server.

After the conference has been initiated, the sensory data is collected (at 725) and transmitted to the agent’s device via the backend server or the cloud computing service (at 730).

The PPG waveform shape is compared against an expected/desired shape to determine if the pulse and blood oxygen saturation is reliable (at 735). For example, in some embodiments there may be a change in color in the collected PPG waveform depending on if it is a “good” signal versus a “bad” signal. As noted herein, a “good” signal is defined as a signal that matches an exemplary “saw toothed” waveform by a configured degree of accuracy. A mathematical algorithm is employed to determine the match between the prototypical waveform and that of the collected waveform. The algorithm may include, for example, a least means squared function. In some embodiments, other collected data may be leveraged to determine if other collected physiological data is reliable. For example, accelerometer data may indicate if a blood pressure measurement is reliable, whereas a skin conductivity measurement may indicate if an ECG signal can be considered reliable.

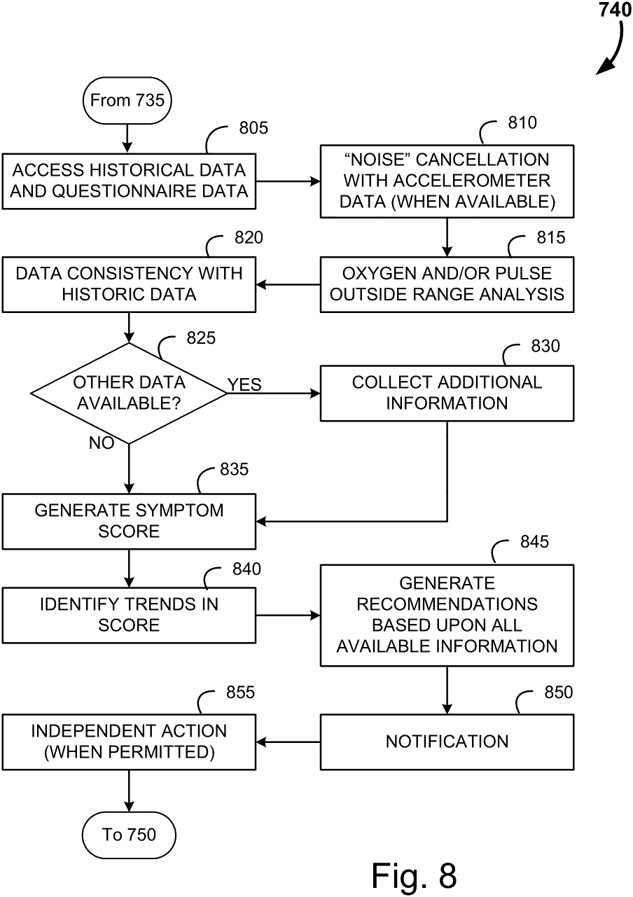

If the data isn’t reliable, it may be discarded (at 745) and the system may simply continue collecting more data. However, reliable data may be subjected to various data analytics (at 740). FIG. 8 provides a more detailed diagram for the example process of data analytics. This process begins with the access not only of the instant collected data, but also access to historical data and profile/questionnaire data (at 805). The collected data may be subjected to transforms/normalizations based upon sensor types, time of day, ambient temperature, which agent is providing instructions, and importantly by motion sensory data (e.g., accelerometers) when available (at 810). In some embodiments, the pulse rate and blood oxygen saturation measurements are compared against target ranges (at 815). A check is made if these collected measurements are consistent with historically collected data (at 820). Significant deviations of collected data versus the historical data could indicate a sensor malfunction or could be indicative of a pathology.

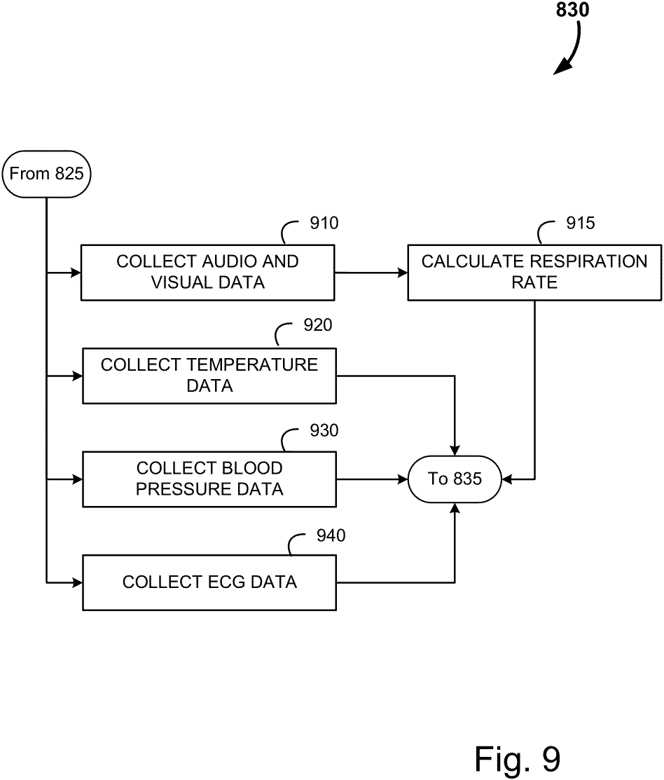

The system may also determine if other data types (e.g., blood pressure, etc.) is available (at 825) and if so, collect this other data as well (at 830). This collected data is illustrated in greater detail in relation to FIG. 9. Here audio and visual data of the patient can be collected (at 910). Generally, the device used to communicate with the healthcare worker, which is running the application, includes a camera and microphone. These may be employed to collect this data. The patient’s respiration rate may be calculated using this audio and visual data (at 915) using ML models. The visual data that is collected may also be employed to identify the kind of exercise the patient is engaged in and identify if the exercise is being done correctly. This determination is made by mapping musculature/bone alignment and comparing the motions of the patient against template movements for each exercise type. This allows the healthcare worker to be notified that the exercise is being performed incorrectly, and/or provides feedback directly to the patient so that she may improve her form. In addition, or in the alternative, to collecting audio and visual data, temperature data (at 920), blood pressure data (at 930) and ECG data (at 940) can be collected.

Returning to FIG. 8, all collected data may be employed to generate the symptom score (at 835) as previously discussed. Additionally, trends in the score (and other physiological data) may be identified, and predictions extrapolated (at 840). Recommendations for treatment may be generated based upon the collected and historical data (at 845). Again, such recommendations may be rule based, or employ ML models. Generally, the recommendations are designed to alter the activity level of the patient in order to bring the heartrate and oxygen saturation levels within a target range. The range may vary based upon profile information (e.g., age, gender, etc.) as well as historically collected data. Additionally, blood pressure and other collected data may also be shifted into a desired range(s) by altering activity levels, supplemental oxygen levels, and the like.

As noted before, recommendations may include the changing directly (or through a recommendation) of supplemental oxygen levels, and even an automated agent. The virtual agent may employ the patient’s heartrate, blood oxygen level and computer vision collected by a camera, to detect how the individual is exercising, and how to change or moderate the intensity of the exercise or alter the amount of supplemental oxygen (when applicable).

All recommendations may be output directly to the patient and/or to the healthcare worker as a notification (at 850). Notifications may appear as alerts to the healthcare worker in some embodiments or may even take the form of instructions to the patient from a virtual agent. In some embodiments, the system may even be configured to take independent action (at 855) such as automatically adjusting settings on exercise equipment (such as changing the settings on Therabands or other calibrate-able equipment) or adjusting supplemental oxygen levels.

Returning to FIG. 7, once the data analytics has been completed, an inquiry is made if the activity level (or other metric) for the patient requires modification (at 750). If so, the activity levels may be altered (at 755) again through the healthcare worker, with guidance from the system, or via the system directly. A determination is made if the process/therapy session has concluded (at 760). If not, the process starts over again with the collection of additional sensor data.

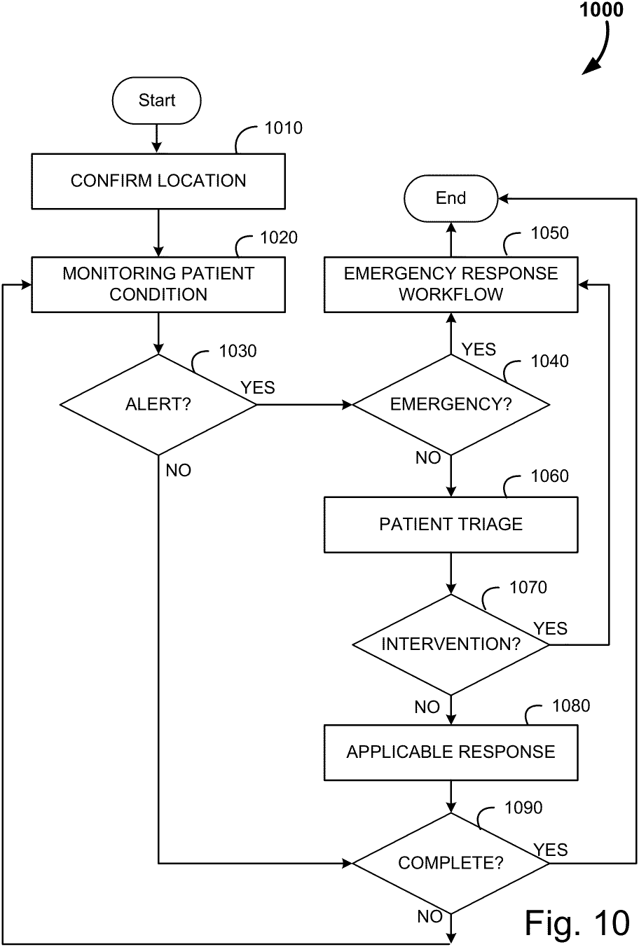

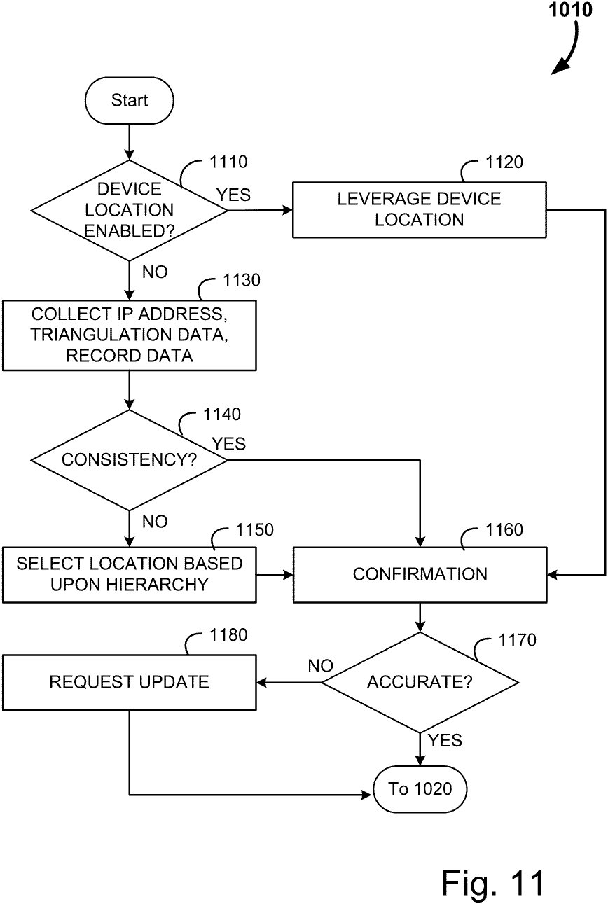

Turning now to FIG. 10, an example process is provided for the delivery of medical and other emergency services to a patient engaged in a remote therapy session, shown generally at 1000. In this example process, the crucial first step is to identify the location of the patient (at 1010). FIG. 11 provides a more detailed example of this particular process. The simplest means for collecting location data is when the device itself is collecting location data (e.g., GPS). A determination of whether the device has location services enabled, and if so, that they are shared with the remote therapy application, is made (at 1110). For some users, the device they are operating on, such as a tablet, may lack the ability to collect location data. For other patients, they may have concerns over privacy, and may have either disabled location services, or may prevent the device from sharing the location.

Regardless of cause, if the location of the device is not readily available, the system may be forced to engage in alternate location determination strategies. These include looking up recorded data of the patient’s residence, and collection of other location indicative data (at 1130). For example, triangulation of cellular tower pings may be leveraged when the device is connected to a cellular network, and lookup of IP addresses may be leveraged for internet connected devices. The system checks then for consistency between the collected data, and the patient records (at 1140). If there is no consistency, the system must select the ‘best guess’ of the patient’s location based upon ML analysis and/or a hierarchical structure. For example, an ML analysis may leverage the background of the user to compare it against a stored background of their prior sessions. When these images are a match within a particular confidence threshold, the last known address may be leveraged. In another embodiment, when the image analysis shows the user in a hotel room (which are of relative standardized layouts), the system may instead utilize an IP address. In a non-ML process based upon hierarchies, cellular triangulation may be utilized first, IP address second, and recorded address third (as these data sources are available). Regardless of how the location is determined, leveraging device location (at 1120), a consensus (at 1140) or by hierarchical or ML estimation (at 1150), at the initiation of the therapy session, the patient may be presented with the exact address (when available), or approximate location for confirmation (at 1160). Confirmation may be as simple as the patient pressing an acknowledgement button on their device’s screen or may require the user to input missing location data. Confirmation may also include verbal confirmation in real-time. If the location data is accurate (at 1170) the process ends. Alternatively, the patient may be requested to input updated location information (at 1180) before the conclusion of this sub-process. Any address information supplied is converted by the system into latitude and longitude coordinates and/or convert it to political entities, such as city, county, state and country.

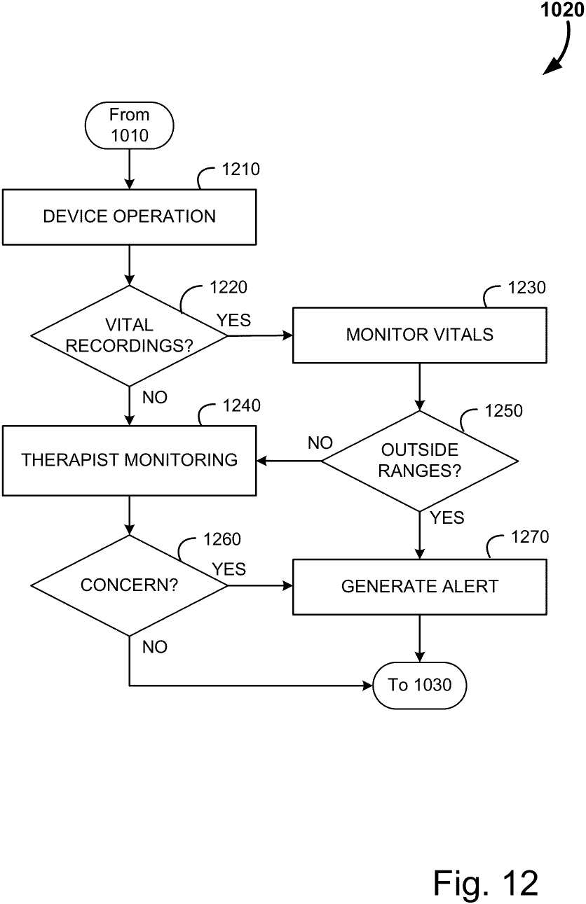

Returning to FIG. 10, after location is confirmed, the patient’s condition is monitored throughout the therapy session (at 1020). FIG. 12 provides a more detailed description of this sub-process of patient monitoring. Initially, the data collection devices begin operation (at 1210). This may include the camera and audio on the patient’s device, as well as ancillary data collection devices as previously discussed (e.g., ECG, O₂ monitor, heartrate monitor, breathing monitors, etc.).

When vitals are being recorded (at 1220) they may be monitored against ‘safe’ levels (at 1230). Safe levels may be set based upon guidance informed by the patient’s race, age, gender, pathologies, and fitness levels (all identifiable using the patient records). Additionally, in some advanced operations, historical information regarding the patient’s vital records may likewise be leveraged to modulate what is determined to be a safe range for the patient for a given physiological measure.

When the vitals that are collected extend outside the allowed ranges for a sufficient duration (at 1250), the system may generate an alert (at 1270). The duration that the vitals must be outside the allowed range(s) may be modulated in order to ensure the patient receives attention when they require it, but without generating unnecessary false positives. As such, this duration is generally dynamic, and depends upon the following factors: vital measurement type, degree of vital measurement from the allowed range, trajectory/changes identified in the vitals. For example, a pulse rate may be susceptible to relatively minor complications if it rises too far above the allowed range, as such the duration that the pulse is above the allowed limit (say 140 bpm for a given patient) may be longer than the duration of a blood oxygenation level below a given threshold (for example 45 seconds for the pulse, and only 15 seconds for the oxygenation levels). Likewise, the degree of difference between the range, and the deviation may modify the allowed duration. Leveraging the prior example, a heartrate of 145 bpm may be tolerated for over a minute, whereas a heartrate of 160 may result in an alert after only 20 seconds. Lastly, trajectory/change in the metric may be used to further modify the time. Leveraging the prior example, a patient whose heart rate is only changing roughly 1 bpm every couple minute, and with a current rate of 145 bpm, may have a duration of two minutes before an alert is generated, whereas when the patient’s heart rate is increasing by 10 bpm every minute may have an alert generated after only 30 seconds.

It should be noted that the above discussion focuses on ‘recorded vitals’. This term is intended to be very broad and include metrics that are derived from less common sources. For example, the video feed may be actively analyzed for events such as a patient falling using ML algorithms. Likewise, speech/audio inputs may be analyzed for words such as “help” or cries of pain. Each of these events would be considered “out of range” for the purposes of this disclosure and would result in the generation of an alert and potentially automatically call EMS and/or connect the patient with an associated medical director, as will be discussed in greater detail.

When vitals are not being recorded, or when the vitals are all within acceptable ranges, the system still ensures that the healthcare worker maintains video and audio connectivity with the patient, and the healthcare worker actively monitors the various patients (at 1240). The healthcare worker identifies the presence of acute distress in the patient’s (fainting or a heart attack for example), or evidence of over-exertion, heat stroke, or similar conditions. When such a concern is identified (at 1260) this may also result in an alert in the form of the healthcare worker selecting an emergency icon associated with the given patient.

Returning to FIG. 10, the system determines if there is the presence of such an alert from the monitoring process (at 1030). If not, the system queries if the therapy session is completed (at 1090). If so, the entire process ends. However, when the therapy session is still ongoing, the system may continue with the monitoring (at 1020).

When an alert does occur, however, the system determines if the event is an emergency (at 1040) or not. The determination if there is an emergency may be based upon the alert that was provided. For example, when the alert is initiated by the healthcare worker, the healthcare worker may have the option of selecting an emergency icon for the patient, or a lesser alert icon. When the alert is automatically generated, the system may utilize heuristic rules to determine when the event is a true emergency or a lower-level concern. A patient falling, as identified by an ML model, or evidence of cardiac arrest from ECG sensors, would cause a true emergency signal to be generated. In contrast, a patient with a heart rate that is slightly elevated for too long would trigger a lower-level alert.

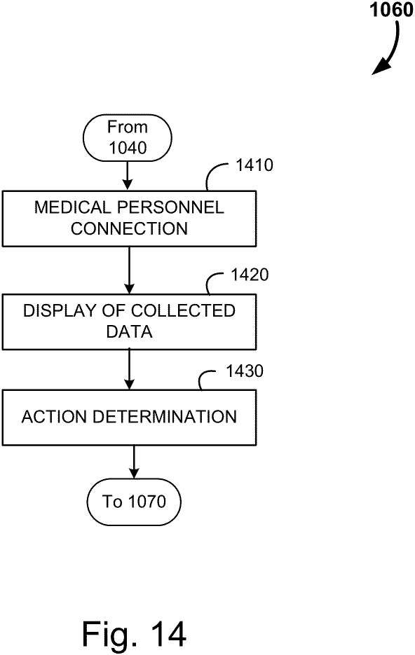

When a true emergency is detected (at 1040) an emergency workflow may be initiated, which will be discussed in greater detail below. However, for lesser level alerts, the system may first focus the attention upon the given patient and a triage event can occur (at 1060). FIG. 14 provides greater detail into this triage process. This triage may be healthcare worker directed and may simply include the healthcare worker asking questions of the patient, observing patient behavior and the like. In some embodiments, triage may include looping in a third-party medical staff for a more formal assessment (at 1410). In some cases, the healthcare worker may be presented the option of whether to involve such a third party.

A third-party medical staff may be on-call for such triage events. This medical professional may generally include a nurse practitioner, physician’s assistant, or a medical doctor. As the disclosed remote therapy systems are highly scalable, with a single healthcare worker potentially treating more than one patient simultaneously, in a similar manner the medical specialist may service dozens of healthcare workers simultaneously. This means that, effectively, the medical specialist could be on call for dozens, if not hundreds of patients at any given time. As emergency responses are generally rare, this results in a situation where a single individual (or small team of individuals) can at any time provide care without compromising safety of the patient cohort. At the same time however, the cost of employing the medical professional is spread amongst many people, effectively reducing the financial costs significantly. Again, as noted before, one of the great benefits of such a remote system is not only improved compliance, frequency and convenience of the therapy, but also making such therapy significantly more cost effective. With these emergency services offerings, the system may indeed also be safer than in-person sessions as medical triage and support may be integrated into the offering.

In the triage, the healthcare worker and/or medical personnel are presented with a live stream of the patient, a recording of the event(s) surrounding the alert, and/or data collected such as vital measurements (at 1420). The action to be taken is then determined by the healthcare worker and/or medical professional (at 1430). It should be noted, that in advanced systems, ML modeling may take the place of a medical professional in this process. The ML model may then present a recommendation of action to the healthcare worker, and the healthcare worker may either adopt or reject the recommendation. These selections by the healthcare worker become a valuable positive and negative training set for further refining ML model operation.

Returning to FIG. 10, the triage process is used to determine when the event being analyzed rises to an emergency level, or whether lesser interventions are needed (at 1070). Lesser interventions may include mere modification of the therapies being offered, having the patient sit or lie down, get an icepack, drink fluids, or alter behaviors in a manner that causes their condition to normalize (at 1080). After such an intervention, the therapy may be completed, or resume (at 1090).

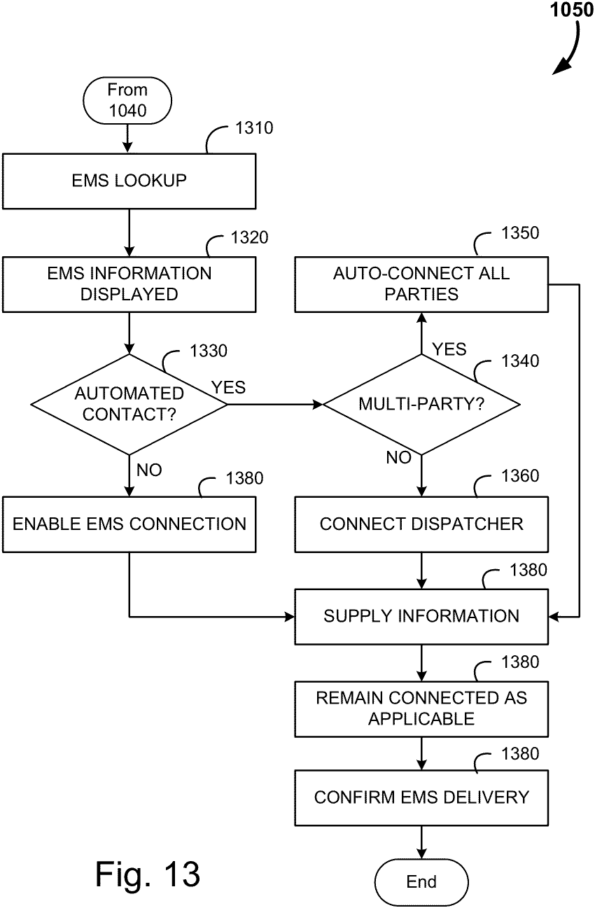

However, when an emergency is identified originally, or after the triage event, the system enters an emergency response workflow (at 1050). The emergency response is presented in considerably more detail in relation to FIG. 13. In this process, a database of EMS contact information is retained and regularly updated for the entire US (and other countries and territories as such information is available). This EMS data provides, at a minimum, direct and indirect contact information, based upon geography, for local EMS providers. Often this EMS information is for local 911 dispatch services, but may likewise include direct hospital contact information, direct police or fire department contact information, as well as public and/or private ambulatory services. The initial step is to leverage the collected location information for the patient and use the EMS database to identify the contact information that is most relevant via a lookup operation (at 1310). This information is presented to the healthcare worker and/or the patient herself (at 1320). A determination is made if the system has the capability and the authority to contact the relevant EMS directly (at 1330). If so, the system may initiate such connection (at 1370).

When the system cannot or is not allowed to engage in direct connectivity with the emergency services, another query is made whether to connect in additional parties besides just the healthcare worker (at 1340). As discussed before, there may be a medical provider that supports the healthcare workers, and through them the patients. It may be advantageous to include the medical personnel into the session upon the detection of an emergency event. This individual may assist in care while the emergency responders are in transit, and likewise assist in describing the emergency to the EMS upon connection. When it is possible to involve medical personnel, this party is connected through the platform such that they have the same data as is available to the healthcare worker (e.g., audio and video connectivity, collected vital information, patient records, etc.). The patient may likewise be connected to the medical specialist via a video connection such that the patient can focus on an ‘authority figure’. This can provide some degree of calming effect for the patient and increases their likeliness of comporting to directives. For example, the medical specialist may tell the patient to lie on their back and elevate their knees, or some other directives. At the same time as connecting in the third-party medical specialist, the system may select and connect the appropriate EMS (at 1350). This uses the lookup of EMS by location of the patient. If a medical professional is not available, the system may simply connect the healthcare worker and patient directly with a dispatcher or other EMS (at 1360).

For any connection process, a decision may be made by the system which EMS contact to connect to. Most often, where there is a 911 dispatch system available the system will prioritize this contact. However, if there is an issue getting through to a higher priority contact, the system may descend through the different EMS contacts until the best contact is reached. The determination of which order of EMS contacts should be in may be based upon contact type and proximity to the patient. For example, if the fire department is directly around the corner from the patient, then this contact may be prioritized just after the dispatcher. Likewise, a very close hospital may be prioritized, and then private ambulatory services. Lastly, as most emergencies are health related, the police may be a last resort to contact.

After the EMS connection is made, the system and individuals involved may provide the EMS with information (at 1380) regarding the patient’s location, current status, nature of the emergency and when appropriate, pertinent medical history (to the extent such information is held in the system’s databases) and any other pertinent and available data. For example, the system may have access to the patient’s pathologies that are the cause for the therapy in the first place. The system may likewise have access to information regarding medications taken for these pathologies. Ideally, such information is provided by the patient upon the setup of their profile in the remote therapy system. Of course, more, or less, information may be available based upon the patient’s willingness to share such information with the system, and the system’s capacity to retain such information.

In some embodiments, this data is supplied to the medical professional and/or healthcare worker via their devices, in order to relay the information to the EMS. In other situations, as the systems become more integrated, the remote therapy system may push notification data directly to the dispatcher’s systems. Such a direct peer-to-peer data transfer provides the fastest and most efficient ability to get potentially life saving information to the EMS.

Even after the EMS has all applicable information, there are physical restraints on how quickly the first responders can actually get to the patient’s location. During this interim period, the EMS contact, medical professional (when present) and healthcare worker may remain connected and in communication with the patient (at 1390). Keeping contact with the patient allows the EMS to become aware of changes in the condition of the patient (which could be helpful, for example, when the paramedics arrive). Keeping contact open also has the advantage of allowing the EMS and/or medical professional to keep instructing the patient in actions that can best improve their outcomes. And likewise, having someone ‘there’ with the patient can have a calming effect, hopefully keeping the patient from panicking and making the condition worse.

Upon arrival of the paramedics (or other EMS personnel), the system may confirm delivery of the care (at 1395) and allow the healthcare worker and/or medical professional to be disconnected from the session with the patient. The system may log the entire process for later review if needed.

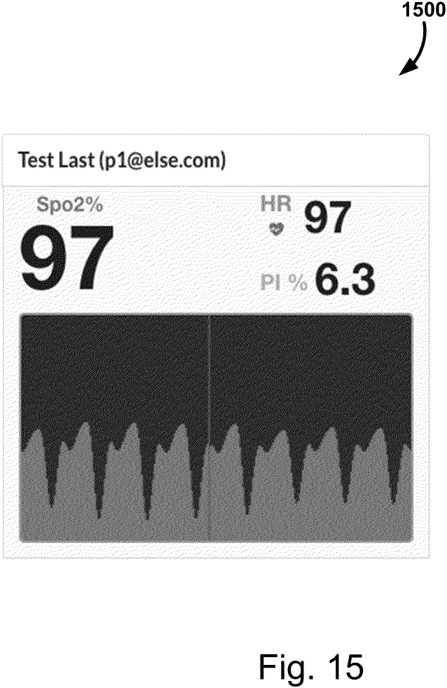

Turning now to FIG. 15, a screenshot of one embodiment of the display on the agent’s device is provided at 1500. Again, the percentage blood oxygen saturation is displayed, along with the heartrate and perfusion index (PI). Additionally, the PPG signal is presented. Note the “saw-tooth” shape to the waveform, indicating that the signals being collected are reliable. In some embodiments, a simple user interface element may indicate the accuracy of the collected physiological information using the algorithms previously discussed. This indicator may be as simple as color coding the illustrated values. For example, when the system determines the recorded signals are very accurate, they may be displayed in a green color. When accuracy is in question, however, they may be displayed in a red color. In some embodiments an intermediate color may indicate signals that have questionable accuracy. Of course, other user interface indications may be employed, including an entirely separate display icon, sounds, vibrations, or the like.

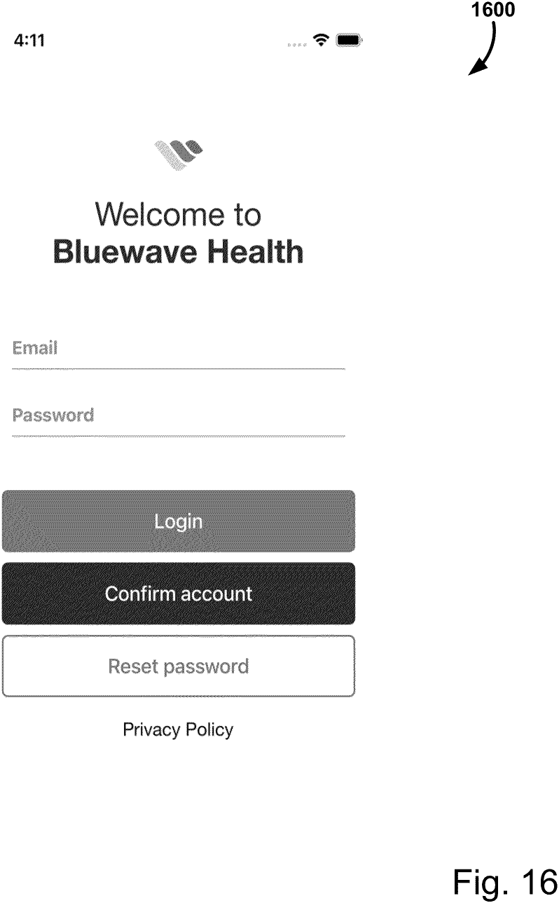

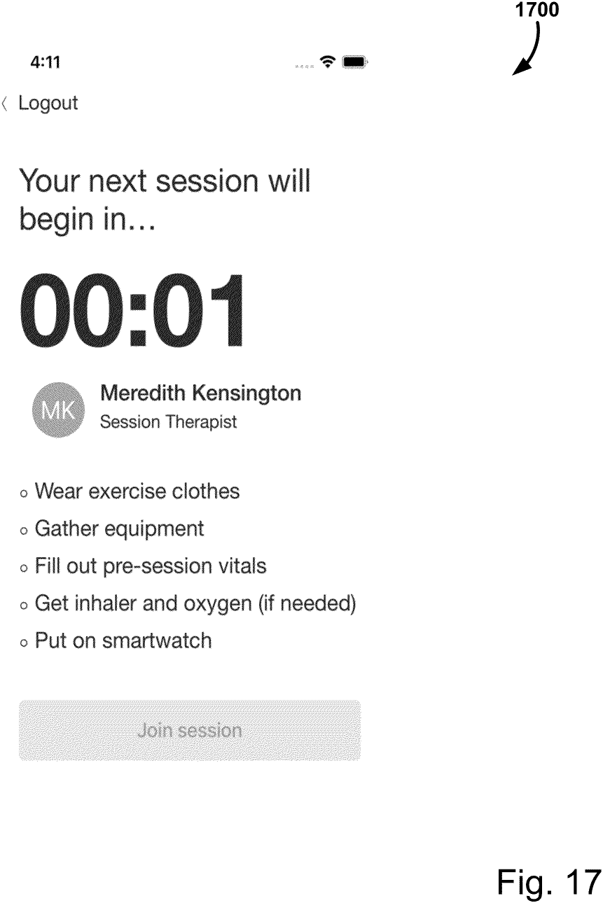

Turning to FIG. 16, an example screenshot of the login screen for the patient is presented at 1600. Typically, a username and password combination are necessary to access the system. It is also possible to generate and confirm a new account, reset the patient’s password, and access the privacy policy from this screen. Once logged in, the patient may be directed to a landing page, as seen at 1700 of FIG. 17. This page indicates to the patient the timing before the therapy session is to start, the name of the agent, and general guidelines for a successful therapy session. Additionally, on this landing page or on a separate screen (not illustrated) historical data for the patient may be displayed, and/or recommendations for the patient, and/or the results of some analytics.

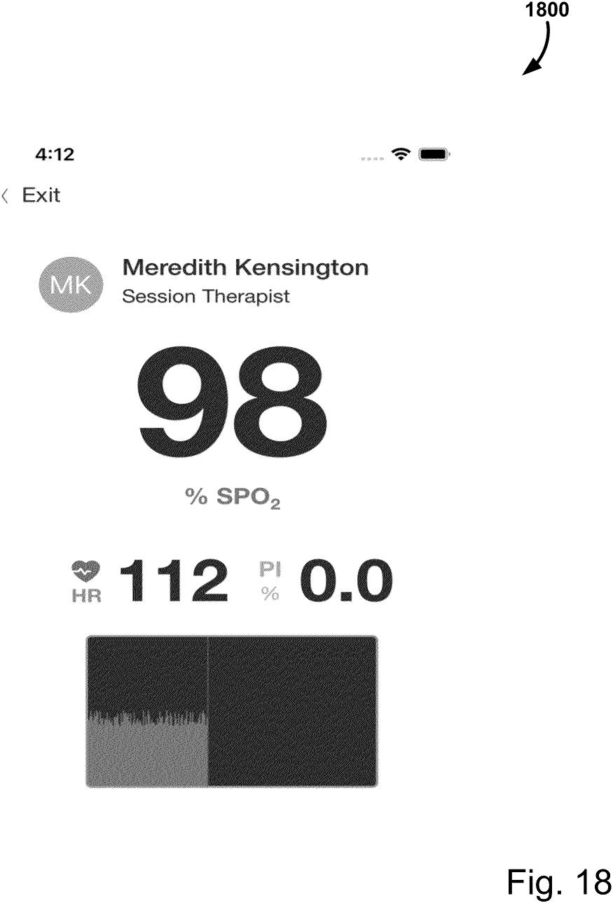

Once the therapy begins the patient may be presented with a screen showing the collected sensory data, as seen at 1800 of FIG. 18. Again, blood oxygenation, heartrate, PI percentage and PPG signals are shown. In some embodiments, a window with the video conference with the agent (or virtual avatar) may also be displayed. On the agent’s side, in addition to the collected sensory data, a video conference screen is provided. Additionally, notifications may be presented as pop-up alerts.

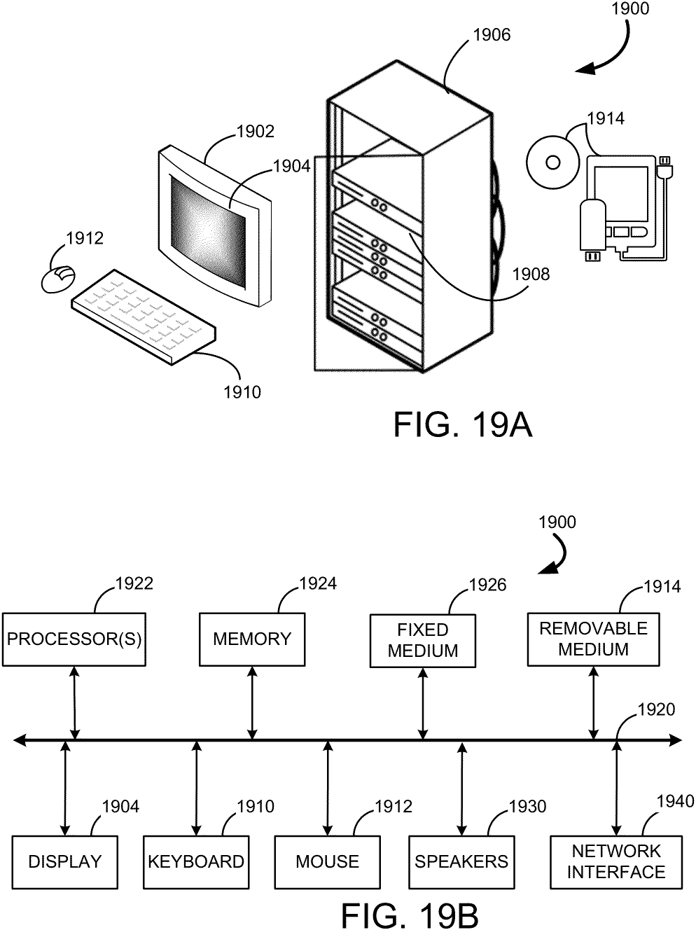

Now that the systems and methods for improving exercise therapy for respiratory conditions and similar pathologies has been discussed in considerable detail, attention shall now be focused upon apparatuses capable of executing the above functions in real-time. To facilitate this discussion, FIGS. 19A and 19B illustrate a Computer System 1900, which is suitable for implementing embodiments of the present invention. FIG. 19A shows one possible physical form of the Computer System 1900. Of course, the Computer System 1900 may have many physical forms ranging from a printed circuit board, an integrated circuit, and a small handheld device up to a huge super computer. Computer system 1900 may include a Monitor 1902, a Display 1904, a Housing 1906, server blades including one or more storage Drives 1908, a Keyboard 1910, and a Mouse 1912. Medium 1914 is a computer-readable medium used to transfer data to and from Computer System 1900.

FIG. 19B is an example of a block diagram for Computer System 1900. Attached to System Bus 1920 are a wide variety of subsystems. Processor(s) 1922 (also referred to as central processing units, or CPUs) are coupled to storage devices, including Memory 1924. Memory 1924 includes random access memory (RAM) and read-only memory (ROM). As is well known in the art, ROM acts to transfer data and instructions uni-directionally to the CPU and RAM is used typically to transfer data and instructions in a bi-directional manner. Both of these types of memories may include any suitable form of the computer-readable media described below. A Fixed Medium 1926 may also be coupled bi-directionally to the Processor 1922; it provides additional data storage capacity and may also include any of the computer-readable media described below. Fixed Medium 1926 may be used to store programs, data, and the like and is typically a secondary storage medium (such as a hard disk) that is slower than primary storage. It may be appreciated that the information retained within Fixed Medium 1926 may, in appropriate cases, be incorporated in standard fashion as virtual memory in Memory 1924. Removable Medium 1914 may take the form of any of the computer-readable media described below.

Processor 1922 is also coupled to a variety of input/output devices, such as Display 1904, Keyboard 1910, Mouse 1912 and Speakers 1930. In general, an input/output device may be any of: video displays, track balls, mice, keyboards, microphones, touch-sensitive displays, transducer card readers, magnetic or paper tape readers, tablets, styluses, voice or handwriting recognizers, biometrics readers, motion sensors, brain wave readers, or other computers. Processor 1922 optionally may be coupled to another computer or telecommunications network using Network Interface 1940. With such a Network Interface 1940, it is contemplated that the Processor 1922 might receive information from the network, or might output information to the network in the course of performing the above-described remote therapy and emergency response activities. Furthermore, method embodiments of the present invention may execute solely upon Processor 1922 or may execute over a network such as the Internet in conjunction with a remote CPU that shares a portion of the processing.

Software is typically stored in the non-volatile memory and/or the drive unit and/or stored in the cloud. Indeed, for large programs, it may not even be possible to store the entire program in the memory. Nevertheless, it should be understood that for software to run, if necessary, it is moved to a computer readable location appropriate for processing, and for illustrative purposes, that location is referred to as the memory in this disclosure. Even when software is moved to the memory for execution, the processor may typically make use of hardware registers to store values associated with the software, and local cache that, ideally, serves to speed up execution. As used herein, a software program is assumed to be stored at any known or convenient location (from non-volatile storage to hardware registers) when the software program is referred to as “implemented in a computer-readable medium.” A processor is considered to be “configured to execute a program” when at least one value associated with the program is stored in a register readable by the processor.

In operation, the computer system 1900 can be controlled by operating system software that includes a file management system, such as a medium operating system. One example of operating system software with associated file management system software is the family of operating systems known as Windows® from Microsoft Corporation of Redmond, Washington, and their associated file management systems. Another example of operating system software with its associated file management system software is the Linux operating system and its associated file management system. The file management system is typically stored in the non-volatile memory and/or drive unit and causes the processor to execute the various acts required by the operating system to input and output data and to store data in the memory, including storing files on the non-volatile memory and/or drive unit.

Some portions of the detailed description may be presented in terms of algorithms and symbolic representations of operations on data bits within a computer memory. These algorithmic descriptions and representations are the means used by those skilled in the data processing arts to most effectively convey the substance of their work to others skilled in the art. An algorithm is, here and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

The algorithms and displays presented herein are not inherently related to any particular computer or other apparatus. Various general purpose systems may be used with programs in accordance with the teachings herein, or it may prove convenient to construct more specialized apparatus to perform the methods of some embodiments. The required structure for a variety of these systems may appear from the description below. In addition, the techniques are not described with reference to any particular programming language, and various embodiments may, thus, be implemented using a variety of programming languages.

In alternative embodiments, the machine operates as a standalone device or may be connected (e.g., networked) to other machines. In a networked deployment, the machine may operate in the capacity of a server or a client machine in a client-server network environment or as a peer machine in a peer-to-peer (or distributed) network environment.

The machine may be a server computer, a client computer, a personal computer (PC), a tablet PC, a laptop computer, a set-top box (STB), a personal digital assistant (PDA), a cellular telephone, an iPhone, a Blackberry, Glasses with a processor, Headphones with a processor, Virtual Reality devices, a processor, distributed processors working together, a telephone, a web appliance, a network router, switch or bridge, or any machine capable of executing a set of instructions (sequential or otherwise) that specify actions to be taken by that machine.

While the machine-readable medium or machine-readable storage medium is shown in an exemplary embodiment to be a single medium, the term “machine-readable medium” and “machine-readable storage medium” should be taken to include a single medium or multiple media (e.g., a centralized or distributed database, and/or associated caches and servers) that store the one or more sets of instructions. The term “machine-readable medium” and “machine-readable storage medium” shall also be taken to include any medium that is capable of storing, encoding or carrying a set of instructions for execution by the machine and that cause the machine to perform any one or more of the methodologies of the presently disclosed technique and innovation.

In general, the routines executed to implement the embodiments of the disclosure may be implemented as part of an operating system or a specific application, component, program, object, module or sequence of instructions referred to as “computer programs.” The computer programs typically comprise one or more instructions set at various times in various memory and storage devices in a computer (or distributed across computers), and when read and executed by one or more processing units or processors in a computer (or across computers), cause the computer(s) to perform operations to execute elements involving the various aspects of the disclosure.

Moreover, while embodiments have been described in the context of fully functioning computers and computer systems, those skilled in the art will appreciate that the various embodiments are capable of being distributed as a program product in a variety of forms, and that the disclosure applies equally regardless of the particular type of machine or computer-readable media used to actually effect the distribution

While this invention has been described in terms of several embodiments, there are alterations, modifications, permutations, and substitute equivalents, which fall within the scope of this invention. Although sub-section titles have been provided to aid in the description of the invention, these titles are merely illustrative and are not intended to limit the scope of the present invention. It should also be noted that there are many alternative ways of implementing the methods and apparatuses of the present invention. It is therefore intended that the following appended claims be interpreted as including all such alterations, modifications, permutations, and substitute equivalents as fall within the true spirit and scope of the present invention.

What is claimed is:

1. A computerized method for providing therapy guidance to an agent, the method comprising:
collecting sensory data and photoplethysmography (PPG) data from a patient;
identifying the sensory data when the PPG data differs from a prototypical waveform pattern by a configured threshold;
analyzing the sensory data that was not identified to generate a recommendation; and
supplying the recommendation to at least one of a patient, a clinician, and an agent.

2. The method of claim 1, wherein the sensory data includes pulse rate and blood oxygen saturation.

3. The method of claim 1, wherein the sensory data includes at least one of a perfusion index (PI) percentage, blood pressure, electrocardiogram (ECG) signals, audio and video signals, and temperature.

4. The method of claim 1, wherein the recommendation is generated using historical patient profile and sensory data.

5. The method of claim 1, wherein the recommendation is provided to the agent as an alert.

6. The method of claim 1, wherein the agent is a virtual agent.

7. The method of claim 1, wherein the prototypical waveform pattern is a saw-toothed shape.

8. The method of claim 1, wherein the PPG data differing from the prototypical waveform pattern is calculated by a least means squared algorithm.

9. The method of claim 1, wherein the recommendation is generated by a set of rules comparing the collected sensory data to target values.

10. The method of claim 1, wherein the recommendation is generated by machine learning (ML) models and/or algorithms.

11. A computerized method for optimizing the activity level of a patient, the method comprising:
collecting sensory data and photoplethysmography (PPG) data from a patient;
transmitting the collected sensory data and PPG data to an agent;
filtering the collected sensory data responsive to the PPG data to generate accurate sensory data;
receiving a recommendation from the agent based upon the accurate sensory data; and
modifying the patient’s activity level responsive to the recommendation.

12. The method of claim 11, wherein the sensory data includes pulse rate and blood oxygen saturation.

13. The method of claim 11, wherein the sensory data includes at least one of a perfusion index (PI) percentage, blood pressure, electrocardiogram (ECG) signals, audio and video signals, and temperature.

14. The method of claim 11, wherein the recommendation is generated using historical patient profile and sensory data.

15. The method of claim 11, further comprising providing the agent a notification.

16. The method of claim 15, wherein the notification indicates accuracy of the sensory data.

17. The method of claim 16, wherein the notification is based upon the PPG data.

18. The method of claim 17, wherein the PPG data is compared against a known waveform pattern and accuracy of the sensory data is dependent on the degree of similarity between the PPG data and the known waveform pattern.

19. The method of claim 18, wherein the peaks and valleys of the known waveform pattern are fit against the PPG data, the known waveform pattern amplitude is normalized to the PPG data and a least means squared function is applied between the normalized known waveform pattern and the PPG data.

20. The method of claim 19, wherein the applied least means squared result above a set threshold triggers a notification that the sensory data may be inaccurate.
